(19) United States
(12) Patent Application Publication
(10) Pub. No.: US 2007/0162047 A1
(43) Pub. Date: Jul. 12, 2007

(54) Apparatus and method for colonoscopic appendectomy

(76) Inventor: Anke Gasche, Klosterneuburg (AT)

Correspondence Address: ROGITZ & ASSOCIATES, 750 B STREET, SUITE 3120, SAN DIEGO, CA, 92101, US

(21) Appl. No.: 10/549,599

(22) PCT Filed: Mar. 17, 2004
(86) PCT No.: PCT/EP04/02829

§ 371 Date: Jan. 3, 2007

Related U.S. Application Data
(60) Provisional application No. 60/455,486, filed on Mar. 18, 2003.

(51) Int. Cl. A61B 17/26

(52) U.S. Cl. 606/113

(57) ABSTRACT

A kit for performing removal of a vermiform appendix (20) from a patient comprises an elongated flexible element (140) extending between a proximal end and a distal end, means (150) attached to the distal end of the elongated flexible element for anchoring the distal end to interior walls of a central lumen (26) defined by the appendix when the distal end is disposed within the central Lumen, a tubular element (100) defining a central Lumen adapted for receiving a colonoscope (90), and means for ligating or cutting (168) the appendix at the appendix Base after the appendix is inverted into the cecum (12). A method for performing removal of a vermiform appendix from patient comprises positioning a colonoscope into the cecum of the patient, irrigating the central lumen of the appendix, guiding a distal end of an elongated flexible element into the central lumen of the appendix, wherein the flexible element comprises anchoring means anchoring the distal end to the interior walk of the appendix, providing counterforce against the appendix base, pulling the elongated flexible element toward the cecum, wherein the elongated flexible element draws the appendix into the cecum, and ligating or cutting the appendix.

### FIELD OF THE INVENTION

The present invention relates to apparatuses and methods for performing appendectomy, more particularly, to apparatuses and methods for performing colonoscopic appendectomy.

### BACKGROUND OF THE INVENTION

The vermiform appendix (VA) is a narrow, worm-shaped tube that extends from the posteromedial wall of the cecum. Appendicitis is a term for condition characterized by obstruction and inflammation of the vermiform appendix, which may lead to infection, thrombosis, necrosis, and perforation.

Open or laparoscopic appendectomy (also called appendicectomy) is the current treatment for acute simple appendicitis if adequate facilities and qualified personnel are available. Although appendicitis may resolve without operation, a policy of non-operative treatment is regarded hazardous because delay risks perforation. Little preparation for operation is necessary in young adults with simple appendicitis. Children require fluid and electrolyte repletion, and old patients need evaluation and treatment of associated diseases. Evidence supports the use of systemic antibiotics (e.g., intravenous metronidazole and ceftizoxime) to prevent wound infection in simple appendicitis.

Appendectomy is mandatory for perforated appendicitis with spreading or generalized peritonitis. If a mass is palpable or visible on imaging studies, initial treatment may be either operative or non-operative. Operation resolves the problem promptly in most cases, but operation for appendiceal abscess can be difficult and there is risk of spreading a localized infection into other parts of the peritoneal cavity. Expectant management with intravenous fluids and antibiotics is favored by some surgeons for this reason. The mass may resolve completely, or an abscess may develop. Percutaneous CT-guided drainage of an abscess, if it is possible, allows the acute inflammation to be resolved.

Presently, open appendectomy is mostly done by simple surgical removal of the vermiform appendix. The standard surgical approach is a skin- and muscle-splitting incision about 7 cm in length in the right lower abdominal quadrant. The main blood vessels within the mesoappendix are clamped off and the appendix is cut away after ligation at its base. The resulting stump, which has a small opening and therefore carries the risk of bacterial contamination of the peritoneal cavity, is further inverted into the cecum and closed by a suture. Finally the muscle layers and then the skin are sewn together.

Adverse events of open appendectomy include infectious complications (wound infection, intra-abdominal abscess), postoperative pain, colonic perforation, side effects from general anesthesia, and scar formation (skin and peritoneum), which may cause small bowel obstruction later in life. Also, the procedure is associated with hospitalization.

Laparoscopic appendectomy is a recent innovation, and it is rapidly becoming widely available, as surgeons have learned the technique. The success rates are high, and complications are infrequent. The procedure can be safely performed in pregnant women.

When the surgeon conducts a laparoscopic appendectomy, four incisions, each about 2 cm in length, are made. One incision is near the umbilicus, and one is between the umbilicus and the pubic bone. Two other incisions are smaller and are in the right side of the lower abdomen. The surgeon then passes a camera and special instruments through these incisions. With the aid of this equipment, the surgeon visually examines the abdominal organs and identifies the appendix. Similarly to open appendectomy, the appendix is freed from all of its attachments, removed, and retrieved through one of the incisions. The instruments are removed and then all of the incisions are closed.

Compared to open appendectomy, laparoscopic appendectomy halves the number of wound infections, and reduces pain on the first postoperative day, duration of hospital stay, and time to return to work, but at the cost of a threefold increase in the number of postoperative intra-abdominal abscesses. Possibly, the increase in abscesses may be obviated if laparoscopic appendectomy is avoided in patients with probable perforated appendicitis.

As an alternative, the inversion of the appendix has been developed in the prior art as a distinct surgical technique. Although this prior art procedure requires an open surgical field, it is possibly safer than the above described procedures, because the lumen of the bowel is not opened, and this, in turn, reduces the risk of infection. This procedure begins also with a skin- and muscle-splitting incision of about 7 cm in length. Next, the mesoappendix is ligated and stripped. Then the appendix is inverted either at the tip or at its base until it is inside out, and located within the cecum. A suture is then used to maintain intussusception of the vermiform appendix. Because of the loss of blood supply, the appendix is thought to become necrotic and to be lost to the feces during the following days. In rare cases, however, the inverted vermiform appendix has been found in loco many years after the operation. Because this procedure does not carry the risk of serious infection from excision of the appendix, it has been propagated for prophylactic appendectomy during abdominal surgery for other reasons than appendicitis. It is performed specifically in women who are undergoing laparotomy because of obstetrical or gynecological disease including cesarian section, ectopic pregnancy or myomectomy.

One type of conventional device for inversion appendectomy uses a surgical probe or a blunt end of a large Keith needle to invert the tip of the appendix from outside of the appendix into itself and into the cecum. A hemostatic stitch of nonabsorbable material is placed in the wall of the cecum at the attachment of the mesoappendix to occlude the blood supply to the appendix in the wall of the cecum. This same stitch approximates the taenia coli to hold the appendix within the cecum. The infarcted appendix will slough off in a few days.

The conventional devices and procedures for appendectomy require skin cuts and surgical operations in the patient's abdomen, and cause surgical trauma. Moreover, the conventional procedures are complicated and are expensive. Therefore, there is a need for a specially designed surgical procedure and device for inversion appendectomy.

It is an object of the present invention to provide a specially designed surgical device and method for inversion appendectomy. It is a further object of the present invention to provide a simplified surgical device and procedure for inversion appendectomy.

### SUMMARY OF THE INVENTION

A vermiform appendix is a narrow, closed ended, worm-shaped tube, which usually extends from an orifice the posteromedial wall of the cecum, about 2 cm below the ileocecal valve. The vermiform appendix may occupy one of several positions: (a) it may lie behind the cecum and the lower part of the ascending colon (retrocecal); (b) it may descend over the brim of the lesser pelvis (pelvic); (c) it may lie below the cecum (subcecal; (d) it may lie in front of the terminal ileum and may then be in contact with the anterior abdominal wall (preiliao; or (e) it may lie behind the terminal ileum (retroilial). The cecum, at the junction of the small and large intestine, is usually in the right iliac fossa and easily reached during colonoscopy. The vermiform appendix varies from 2 to 20 cm in length, the average being about 9 cm. It is longer in the child than in the adult and may atrophy and become smaller after mid-adult life. It is connected by a short mesoappendix to the lower part of the mesentery of the terminal ileum. This fold, in the majority of cases, is more or less triangular, and as a rule extends along the entire length of the tube.

The orifice of the vermiform appendix is typically inspected during colonoscopy. Appendicitis can be diagnosed through colonoscopy in patients with a typical clinical presentations. The invention relates to any colonoscopic procedure or any colonoscopic device that is used to perform appendectomy in a non-inverted, non-perforated appendix without opening of the peritoneal cavity. The procedure is called colonoscopic appendectomy. The invention may also be used without a colonoscope. Preferably, the invention utilizes a vision system, and is referenced to below as endoscopic.

This novel endoscopic procedure is described herein below with reference to instruments and devices, some of which have been developed specifically for the implementation of this procedure. While some of the devices described herein are particularly adapted to this procedure, it is to be understood that commercially available devices may also be used to advantage to implement the method of the invention. Therefore, the endoscopic procedure of the invention is not to be limited to the use of the particular instruments described herein. The provision and use of devices specially adapted to this procedure may, however, facilitate its successful implementation. As will also be appreciated and understood from the disclosure to follow, the instruments developed for the implementation of this procedure may also be used to advantage in the conduct of other medical procedures. Thus, those novel instruments are not to be construed as limited to the uses described herein with reference to colonoscopic appendectomy.

The present invention provides apparatuses and methods for performing colonoscopic appendectomy. As noted above, a typical vermiform appendix extends from the posteromedial wall of the cecum. The vermiform appendix includes an appendix base at the posteromedial wall of the cecum and interior walls defining a central lumen which extends from an orifice at the appendix base toward a closed distal tip of the appendix. In a colonoscopic appendectomy, the vermiform appendix is preferably accessed by a colonoscope, inverted and pulled into the cecum, ligated, and cut by using specifically adapted colonoscopic devices.

In accordance with one aspect of the present invention, an appendectomy assembly comprises an elongated flexible element extending between a proximal end and a distal end. A means is disposed at the distal end of the elongated flexible element for anchoring the distal end of the elongated flexible element to at least one of the interior walls of the lumen of the appendix when the distal end is disposed within the central lumen. By pulling the elongated flexible element backward toward the proximal end, a force is provided for inverting the vermiform appendix inward into the cecum. The elongated flexible element is preferably dimensioned to be adapted for passing through an inner channel of a colonoscope. Alternatively, it may pass through a central lumen of a guidance catheter.

In one preferred embodiment, the assembly further comprises a tubular element extending between a proximal end and a distal end. The tubular element defines at least one inner channel for accommodating the colonoscope. The assembly may further comprise an introducing means, preferably a wire guide, for guiding the elongated flexible element into the central lumen of the vermiform appendix. The elongated flexible element is preferably a catheter disposed over the wire guide and the anchoring means includes at least one balloon disposed at the distal end of the elongated flexible element.

The assembly may comprise a cutting device that can be advanced to the appendix base through an additional inner channel of the tubular element. The cutting device is used for enlarging the orifice or the proximal portion of the central lumen of the appendix.

The assembly may further comprise a ligating means, preferably a loop having a tight tube on it, which can move only in one direction for reducing the size of the loop. The tight tube can be pushed over the loop by a elongated tube of similar diameter, which is connected to and can be operated by the handle. The ligating means can be received by an additional inner channel defined by the tubular element or an inner instrument channel of the colonoscope. The ligating loop is attached to an elongated string or wire passing through the elongated tube and can be detachable from the elongated string or wire after the loop ligates the inverted appendix. Instead of the ligating loop, a clip could be used for ligating the appendix. In one preferred embodiment, the assembly further comprises a second elongated wire which forms a wire loop at a distal end. The second elongated wire is adapted for carrying electrical cautery current for cutting or coagulating the ligated appendix. The second elongated wire also can pass through a inner channel of the tubular element or the colonoscope.

There are numerous embodiments and aspects to exploit the principals of the invention. These include, but are not necessarily limited to, for example, drainage and irrigation of the lumen of the appendix in cases with appendicitis, inverting the vermiform appendix without opening the peritoneal cavity, cutting and removing the appendix and its mesoappendix, sealing of the cecal opening for prevention of infectious complications, and preventing bleeding from the main artery to the appendix.

The procedure preferably starts with advancing a colonoscope into the cecum. Before colonoscopy, a tubular element, preferably a colonic overtube, with a proximal end for location externally of a colon, and a distal end for insertion into a colon, and a colonoscope lumen, or channel, extending therethrough for passing the overtube over a colonoscope, is placed over the colonoscope. The length of this overtube is about 10 to 15 cm shorter than a regular colonoscope. The overtube is typically advanced together with the colonoscope, or advanced over the colonoscope into the cecum after the colonoscope has been positioned in the cecum. When the distal end of the overtube is kept behind the tip of the colonoscope, the colonoscope has full maneuverability. During colonoscopy, air or some other gas is used to insufflate the otherwise collapsed colon.

The overtube is preferably flexible and is fixed in length. The distal end of the overtube has a conical shape to minimize the chance that parts of the colon wall may become trapped between the overtube and the colonoscope during advancement of the overtube. The proximal end of the overtube has a flexible seal that prevents insufflated air from leaking between the colonoscope and overtube. When the air-or the gas is removed by suction through the instrument channel of the colonoscope, this seal also ensures building up of vacuum. The proximal end of the overtube has a handle to ensure good maneuverability and to prevent the overtube from complete insertion into the colon. The inner surface of the overtube comprises a coating of a lubricious material to ensure smooth advancement of the overtube over the colonoscope.

In one preferred form, the overtube includes two additional channels, namely a second channel and a third channel (preferably with a diameter of about 3.2 mm), each having a sealing means at a proximal end at the handle and a distal opening on the inner surface of the distal end of the overtube. The second channel defined within the overtube holds a detachable ligating device, which is preferably a detachable ligating loop. The ligating device is used to close the cecal opening and prevent bleeding after removal of the vermiform appendix. The ligating loop can be loaded into the second channel before the procedure. The loop is placed into an edge at the distal end of the overtube. The third channel of the overtube is used for receiving a cutting device, for example, a sphincterotome or a needle knife device to increase the diameter of the proximal portion of the central lumen of the appendix during the inversion process. The third channel is equipped with an angulation knob that is located at or near the distal opening of the overtube channel (similar to that in duodenoscopes) and can be used to change the angle of the cutting device at the distal end. This angulation knob is controlled by a handle on the proximal end of the third channel through a wire connected with the knob and the handle.

When the colonoscope has reached the cecum, an introducing or guiding means, preferably a wire guide is advanced into the orifice of the vermiform appendix through the channel of the colonoscope. The position of this guiding wire can be checked on a monitor using x-ray imaging. A standard catheter is placed over the wire guide and advanced to the tip of the appendix. First, feces remnants in the appendix are washed away by administering saline solution provided through a channel of the catheter. Feces may appear at the orifice of the appendix and can be later removed through an instrument channel of the colonoscope. When no more feces appears at the orifice of the appendix, a radiologically effective amount of contrast media can be given over the catheter and radiographs can be taken from the appendix. The diameter, length, and location of the vermiform appendix can be seen during fluoroscopy on an x-ray monitor. The appendix is checked for signs of perforation. The mucous layer on the epithelial surface can be washed away using N-acetylcysteine.

If the proximal parts of the appendix show a stricture or do not have an adequate diameter for inverting the appendix, they can be dilated using a balloon. If this does not produce a sufficient diameter, the mucosal, submucosal and muscular layer of the appendiceal orifice and proximal part can be cut using an appropriate sphincterotome or a needle knife. This device may be similar to the type used for endoscopic sphincterotomy at endoscopic retrograde cholangio-pancreaticography (ERCP). To avoid opening of the peritoneal cavity, it is important to direct the cut into the mesoappendix. The appendiceal wall is not cut completely through, but only the mucosal, submucosal and parts of the muscular layer are cut. The adequacy of the enlargement of the appendiceal orifice can be checked with a balloon dilator.

Next, after the proximal parts of the appendix have been dilated or the wall has been partially cut, a device for pulling the appendix into the cecum is advanced into the appeniceal lumen (the central lumen) of the appendix through the instrument channel of the colonoscope. This device may be customized for the length, diameter and location of the appendix. The device is preferably constructed as an elongated flexible element having an anchoring means attached to a distal end of the elongated flexible element. The anchoring means is adapted for anchoring the distal end of the elongated element to at least one of the interior walls of the appendix. The interior walls include side walls of the central lumen and bottom wall at the distal tip of the appendix.

In one preferred embodiment, the elongated flexible element comprises an elongated flexible tube or catheter that holds an inflatable balloon having a skid resistant surface. In one preferred form, the elongated flexible tube defines a central lumen for receiving the guide wire. The distal end of the elongated flexible tube with the balloon may be inserted into the central lumen of the appendix over the guide wire. When the balloon is placed in the appendix, air or another fluid medium is introduced into the balloon through the catheter, inflating the balloon. Preferably the balloon is inelastic, although elastic balloon can be used as well. The pressure can be controlled by a manometer that is placed at the proximal end of the catheter. When a desired working pressure is reached, the catheter is locked to hold that pressure.

After the balloon is inflated, in order to invert the appendix, the handle at the proximal end of elongated flexible element is pulled toward the proximal end of the assembly Preferably at the same time, the overtube is advanced toward the appendix base to produce counterforce around the orifice of the appendix. The pulling of the distal end of the elongated flexible element draws the vermiform appendix completely or partially into the cecum. Any inversion that positions the tip of the appendix inside the orifice of the appendix is sufficient to advance next step in the procedure.

In alternative forms, more than one balloon may be used. The balloons may be constructed with conical shape, cylindrical shape, or other shapes. In multi-balloon embodiments, the balloons may be separately and sequentially inflated or deflated. In another preferred embodiment, the elongated flexible element comprises an elongated flexible catheter receiving an inner elongated element. A plurality of flaps or spines are mounted to the distal end of the elongated flexible catheter for anchoring the distal end of the catheter to the interior walls of the appendix. The flaps each have a knob extending into the lumen of the catheter, and the inner elongated element defines a plurality of depressions at a distal end for engaging with the plurality of knobs for adjusting the angle of the flaps. The tips of the flaps preferably carry a skid resistant structure.

In alternative forms, the flaps may open in a sequential order, for example, the flaps at the base first and continuous opening up to the tip. The flaps may have different length, for example, longer at the base and smaller at the tip.

In alternative form, the device may use a vacuum only for anchoring the elongated flexible element to the interior walls of the appendix. For such an embodiment, the device comprises a catheter with distal openings that are introduced into the appendix. The material of the device has some flexibility but does not collapse when vacuum is produced. The vacuum may be produced at the handle outside of the patient with a syringe, at the proximal part of the device. Vacuum causes the wall of the appendix to attach to the catheter. After the appendix is partially inverted, the proximal openings would detach from the appendix wall. This would cause a release of the vacuum. An inner tube is provided to move toward the tip of the device in order to close those openings, which are not anymore attached to the wall of the appendix during the inversion process. Thereby, the vacuum is kept within the appendix and the inversion can be completed.

In order to increase either the vacuum effect or to increase friction, the openings of the vacuum device may be shaped like suction cups, or the vacuum device is provided with a small rounded or pointy spines on the outside of the distal part of the device, which anchor in the wall of the appendix.

In another alternative form, a set of wires is provided for anchoring the elongated flexible element to the appendix walls and for inverting the appendix. The wires (at least one, but most likely four to eight) are preferably covered by a cover sheet. The wires are mounted on small inner catheter and the distal ends of the wires are fixed to the distal tip of the catheter. The proximal ends of the wires are fixed to a tube, which is positioned between the cover sheet and the inner catheter. After the set of wires is placed in the appendix, the cover sheet is pulled off and the wires are released. The cross section of the wire is preferable flat, e.g. 1 mm×0.3 nm. The wires can be preformed, thereby defining the final shape of the wire after released. When the inner catheter is pulled against the middle tube, the wires are pressed against and engage the wall of the appendix. The appendix is inverted by pulling the wires from the proximal end of the assembly. The wires may be provided with short rounded or pointy spines on the outer surface of the wires in order to increase friction. Also the shape and the number of the wires can be adapted to improve the friction/anchoring of wires in the wall of the appendix.

The wires are preferably self-expandable and are provided with specific waving pattern, e.g., a crisscross pattern to form a tubular mesh configuration, such as in an intravascular stent. The self-expandable force of such a device is variable. The tubular mesh (stent) is highly flexible in its longitudinal axis and is preferably covered with a lubricous sheet. After removing the cover sheet, it has a good expansible force in the radial direction, but there is a considerable (10 to 30%) shortening of the stent during release. For increasing friction, stents can also be combined with short rounded or pointy spines, which are mounted at the crossings of the wires.

The anchoring means may also employ other devices for anchoring the distal end of the elongated flexible element to the interior walls of the appendix, for example, combination of wire and balloon, combination of vacuum and balloon, combination of wire and vacuum, combination of wire, balloon, and vacuum, and combination of flaps (or spines) and vacuum. In the combination of balloon and any other devices, the balloon can be one balloon or multi-balloon, and in the multi-balloon forms, the balloons can be inflated sequentially or simultaneously.

The above described inversion devices are preferably used in procedures performing inversion starting from the base of the appendix to the tip of appendix. The present invention also provides tip-to-base inversion devices and methods. The device may include an inner grabbing device, such as a forceps or the like, a sliding tube received over the grabbing device, and an overtube received over the sliding tube. In the procedure, the grabbing-device and the sliding tube are advanced into the appendix. The grabbing device grabs the tip of the appendix and withdraws the tip into the sliding tube. The sliding tube ensures that the wall of the appendix does not wrinkle up when the tip of the appendix is pulled during the inversion process. The overtube is placed at the orifice of the appendix for applying couterforce to the base of the appendix. The sliding tube would slide into the overtube as a consequence of pulling the inner grabbing device and holding the overtube at the base of the appendix during the inversion. The diameter and length of the sliding tube can be customized. In an alternative form, the sliding tube is constructed as a coated, self-expandable mesh wire stent, such as coated intravascular stent. The coat is of a flexible, lubricous material in order to slide within the appendix. The coated self-expandable mesh wire stent has at least the length of the appendix. It is released from an introducing tube by withdrawing a cover sheet. The advantage of such a coated, self-expandable mesh wire is that the expansion increases the space, into which the tip of the appendix is pulled.

In another preferred embodiment, the elongated flexible element is an elongated flexible tube or catheter that holds a series of inflatable conical balloons, each having the base of the cone at a proximal end of the balloon and the tip of the cone at a distal end of the balloon. In one preferred form, the elongated flexible tube defines a central lumen for receiving the guiding wire. The distal end of the elongated flexible tube with the balloons may be inserted into the central lumen of the appendix over the guiding wire. The balloons are positioned in close proximity to each other. The outer surfaces of the balloons carry a skid resistant structure. The balloons are connected over a first channel within the elongated flexible element. Between the adjacent balloons and at the distal tip of the catheter are small holes that are connected over a second channel of the elongated flexible tube. When the device is placed in the appendix, air or another fluid medium is introduced into the balloons through the first channel, inflating the balloons. Preferably the balloons are inelastic, although elastic balloons can be used as well. The pressure can be controlled by a manometer that is placed at the proximal end of the catheter. When the working pressure is reached, the first channel is locked. The second channel is used to produce a vacuum within the central lumen of the appendix, so that the interior walls of the central lumen stick or are attached to the balloons. A syringe is placed at the proximal opening of the second channel and air is aspirated. The second channel can be locked as well.

After the balloons are inflated and a vacuum is created between the balloons and the interior walls of the central lumen of the appendix, in order to invert the appendix, the handle at the proximal end of elongated flexible element is pulled. Preferably at the same time, the overtube is advanced toward the appendix base to produce counterforce around the orifice of the appendix. The pulling of the distal end of the elongated flexible element draws the vermiform appendix completely or partially into the cecum. Any inversion that positions the tip of the appendix inside the orifice of the appendix is sufficient to advance next step in the procedure.

During the inversion, the mesoappendix with the supplying artery is pulled into the space between the inverted and the non-inverted part of the appendix. This mesoappendix varies in diameter. A thick mesoappendix may cause the inversion to be incomplete. In such a case, the mucosal, submucosal and muscular layer at the inverted proximal part of the appendix can be cut using a needle knife or a similar device that is placed in the third channel of the overtube. An angulation knob that is located at the outlet of the third channel can be used to change the angle of the cutting device at the tip. The clockwise location of the needle knife can be adjusted by rotating the overtube. During the cutting, the pulling on the elongated flexible element and pushing on the overtube are preferably continued. As soon as the appendix can be further inverted, the cutting is finished. The incision is generally superficial and any opening of the peritoneal cavity should be avoided.

During the inversion of the vermiform appendix, a detachable ligating device is placed at the orifice of the vermiform appendix, which now has the mesoappendix and the artery to the appendix at its now inverted outer surface. The ligating device has been loaded within the overtube before the procedure and held in a stable position at the distal end of the overtube. The function of the ligating device is to place a tight loop over the appendiceal orifice for prevention of cecal leakage or bleeding from the artery to the appendix. After this detachable ligating device has been placed and secured, the overtube can be partially retracted to allow, a cutting device, preferably a snare wire loop, to be placed over the inverted vermiform appendix and to electrocut and electrocoagulate the appendix at a point close to the position where the detachable ligating device has been placed. Finally the colonoscope retrieves the removed vermiform appendix by pulling it through the overtube, which is kept in place and used for fast reinsertion of the colonoscope for the purpose of a second look at the site of colonoscopic appendectomy. In order to better observe the inversion process with the colonoscope, the distal end of the overtube can be constructed of a transparent material.

In another preferred embodiment, the device for performing colonoscopic appendectomy includes two colonoscopes, a first one used for operating the elongated flexible element, a second one for providing counterforce at the orifice of the appendix, instead of the overtube. The second colonoscope can be constructed like an overtube having a central lumen to accommodate the first colonoscope. In another preferred embodiment, the device may include only one colonoscope that has at least two instrument channels, a first one for receiving the elongated element and a second one for receiving means for providing counterforce to the orifice of the appendix. The means for providing counterforce can be a ring or tube, or other suitable devices. In these two embodiments, the colonoscopes may have more than one inner channels for passing through ligating means, needle knife, snare wire, and other instrument that is useful during the operation.

In another preferred embodiment, the kit may be introduced not over the instrument channel of the colonoscope but independent of it. The colonoscope is only used for maneuvering the kit into the final position and for visual control during the procedure. The kit may be attached to the colonoscope by string, bands, clips or by using a double overtube.

The invention includes both the procedures described above, and sets of instruments (for example, in the form of kits) in performing the procedures.

### BRIEF DESCRIPTION OF THE DRAWINGS

FIG. 1 is a perspective view of a typical colon with a vermiform appendix extending from a posteromedial wall of a cecum;

FIG. 2 is a perspective view of a device in accordance with one embodiment of the present invention;

FIG. 3 is an enlarged schematic view of a proximal end of the tubular element shown in FIG. 2;

FIG. 4 is a cross-sectional schematic view taken along line A-A of the tubular element shown in FIG. 3;

FIG. 5 is a cross-sectional view of a distal end of the tubular element shown in FIG. 2 taken along axis X-X;

FIG. 6A is a schematic view of a detachable ligating loop in accordance with one embodiment of the present invention;

FIG. 6B is a schematic view of the distal end of the ligating means showing that the ligating loop of FIG. 6A is attached to an elongated tube;

FIG. 6C is a schematic view of the handle of the ligating means shown in FIGS. 6A and 6B;

FIG. 7 is a perspective view of a distal end of the tubular element partially cut away to show the ligating means of FIGS. 6A-6C;

FIG. 8 is a schematic view of the device in accordance with one embodiment of the present invention showing that the device is positioned in a patient's cecum;

FIG. 9 is a schematic view of one embodiment of the present invention showing that a distal end of an elongated flexible element is disposed within the appendix;

FIG. 10A is a schematic view of the distal end of the elongated flexible element, which comprises a balloon catheter in accordance with one embodiment of the present invention;

FIG. 10B is a schematic view of the balloon catheter of FIG. 10A with the balloon inflated;

FIG. 11 is a schematic view of the elongated flexible element comprising the balloon catheter shown in FIGS. 10A and 10B in accordance with one preferred embodiment of the present invention;

FIG. 12 is a schematic view of one embodiment of the present invention showing that the appendix is partially inverted into the cecum;

FIG. 13 is a schematic view of one embodiment of the present invention showing that the appendix is inverted into the cecum;

FIG. 14 is a schematic view of one embodiment of the present invention showing that the appendix is ligated and a cutting means is placed for cutting the appendix in accordance with one embodiment of the present invention;

FIG. 15A is a schematic view of the distal end of the elongated flexible element, which comprises a balloon catheter in accordance with one embodiment of the present invention;

FIG. 15B is a schematic view of the elongated flexible element of FIG. 15A;

FIG. 16A is a schematic view of another preferred embodiment of the elongated flexible element;

FIG. 16B is a schematic view of the distal end of the elongated flexible element of FIG. 16A;

FIG. 16C is a cross-sectional view of the distal end of the elongated flexible element taken along line Y-Y of FIG. 16B, showing that the flaps are closed;

FIG. 16D is a cross-sectional view of the distal end of the elongated flexible element taken along line B-B of FIG. 16B, showing that the flaps are closed;

FIG. 16E is a cross-sectional view of the distal end of the elongated flexible element taken along line Y-Y of FIG. 16B, showing that the flaps are opened;

FIG. 16F is a cross-sectional view of the distal end of the elongated flexible element taken along line B-B of FIG. 16B, showing that the flaps are opened;

FIG. 17A is a schematic view of one embodiment of the distal end of the elongated flexible element employing a vacuum for inverting the appendix;

FIG. 17B is a schematic view of the elongated flexible element of FIG. 17A;

FIG. 17C is a schematic view of another embodiment of the distal end of the elongated flexible element of FIG. 17B;

FIG. 17D is a schematic view of another embodiment of the distal end of the elongated flexible element of FIG. 17B;

FIG. 18A is a schematic view of the distal end of one embodiment of the elongated flexible element employing a set of wires, showing that the wires are covered by a cover sheet;

FIG. 18B is a schematic view of the distal end of the elongated flexible element of FIG. 18A, showing that the wires are released from the cover sheet;

FIG. 18C is a schematic view of the distal end of the elongated flexible element of FIG. 18A, showing that the wires are expanded;

FIG. 18D is a schematic view of the elongated flexible element of FIGS. 18A to 18C;

FIG. 18E is a schematic view of another embodiment of the distal end of the elongated flexible element;

FIG. 18F is a schematic view of another embodiment of the distal end of the elongated flexible element;.

FIG. 19A is a schematic view of another preferred embodiment of the elongated flexible element employing a combination of balloon and vacuum;

FIG. 19B is a schematic view of the elongated flexible element of FIG. 19A;

FIG. 20 is a schematic view of one embodiment of the elongated flexible element employing a combination of balloon and wire;

FIG. 21 is a schematic view of one embodiment of the elongated flexible element employing a combination of balloon, wire, and vacuum;

FIG. 22A is a schematic view of one embodiment of a kit together with an appendix for inverting the appendix in accordance with the present invention;

FIG. 22B is a schematic view of the kit of FIG. 22A, showing that the appendix is partially inverted;

FIG. 23 is a schematic view of another embodiment of the kit for inverting the appendix;

FIG. 24A is a schematic view of a kit in accordance with another preferred embodiment of the present invention;

FIG. 24B shows the kit of FIG. 24A together with a colon and an appendix;

FIG. 24C is a cross-sectional view of a double tube of the kit of FIGS. 24A and 24B, taken along line E-E in FIG. 24A;

FIG. 25 shows one preferred embodiment of the kit for inverting the appendix in accordance with the present invention;

FIG. 26A shows a cross-sectional view of an anal adapter in accordance with one embodiment of the invention, taken along line D-D in FIG. 26B; and

FIG. 26B shows a cross-sectional view of the anal adapter in FIG. 26A, taken along line C-C in FIG. 26A.

### DETAILED DESCRIPTION OF THE PREFERRED EMBODIMENTS

For a better understanding of the invention, the following detailed description refers to the accompanying drawings, wherein preferred exemplary embodiments of the present invention are illustrated and described.

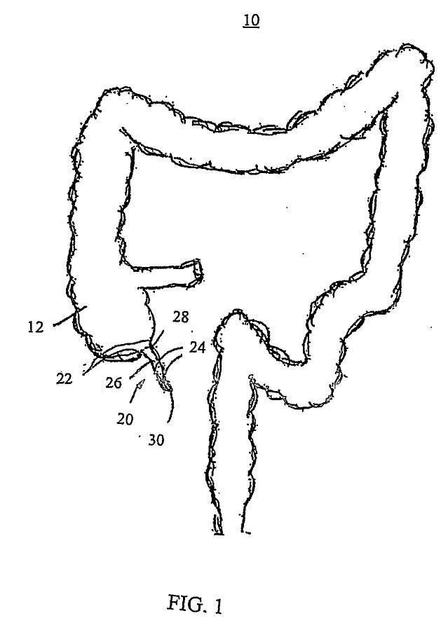

A vermiform appendix is a narrow, worm-shaped tube, which usually extends from the posteromedial wall of a cecum, about 2 cm below the ileocecal valve, and may occupy different positions. A typical colon 10 and a vermiform appendix 20 is illustrated in FIG. 1.

As shown in FIG. 1, the vermiform appendix 20 extends from a base 22 which is connected to the cecum 12. The vermiform appendix 20 includes interior walls 24 defining a central lumen 26 extending from an orifice 28 at the base 22 to a distal tip 30 of the vermiform appendix 20.

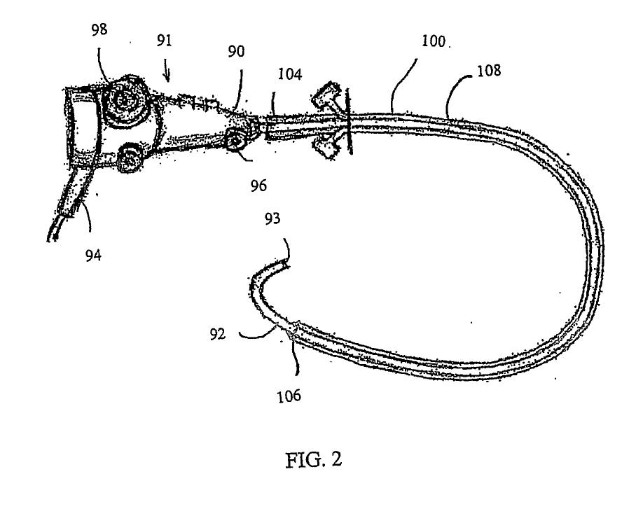

FIG. 2 illustrates a perspective view of a colonoscope 90 and a tubular element 100. The tubular element 100, preferably an overtube, is flexible and includes a proximal end 104 for location externally of a colon, a distal end 106 for insertion into a colon, and a first inner channel 108 extending therethrough for accommodating the colonoscope 90. The length of the overtube 100 is preferably about 10 to 15 cm shorter than a regular colonoscope (e.g. 133 cm or 164 cm, OLYMPUS® CF-100 or CF-100L). The size of the overtube is made to fit certain colonoscopes, including pediatric colonoscopes. The distal end 106 of the overtube 100 is preferably conical shaped to prevent parts of the colon wall from becoming trapped between the overtube 100 and the colonoscope 90 and to avoid damages to the colon wall during advancement of the overtube 100.

The colonoscope 90 includes a head portion 91 and an elongated portion 92. An imaging device, such as a video chip or camera (not shown) is disposed at a distal end 93 of the elongated portion. The colonoscope 90 includes cables 94 for transmitting signals from the imaging device to an external monitor and inner channels with openings 96 at the head portion 91 for passing through surgical tools. A maneuver control handle 98 is disposed at the head portion 91 for positional control of the colonoscope.

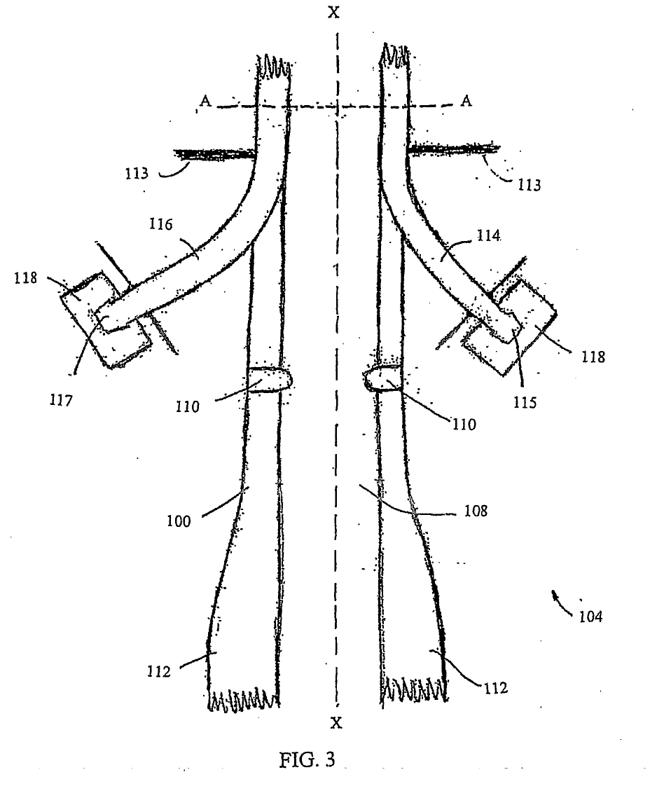

As shown in FIG. 3, the overtube 100 includes a flexible circular seal 110 preferably disposed at the proximal end 104 for sealing the region or chamber formed between the colonoscope 90 and the overtube 100. During colonoscopy, air or some other fluid medium is used to insufflate the otherwise collapsed colon. The seal 110 prevents the insufflated air from leaking between the colonoscope 90 and the overtube 100. When the air or the gas is removed by suction through an instrument channel of the colonoscope 90, the seal 110 also enables to maintain vacuum in the chamber between the colonoscope 90 and the overtube 100. The proximal end 104 of the overtube 100 has a handle 112 for a surgeon to manipulate the overtube 100 and a plate 113 disposed around the overtube 100 to prevent the overtube 100 from complete insertion into the colon. In a preferred embodiment, the inner surface of the overtube 100 comprises a coating of lubricious material to enable smooth advancement of the colonoscope 90 within the inner channel 108 of the overtube 100.

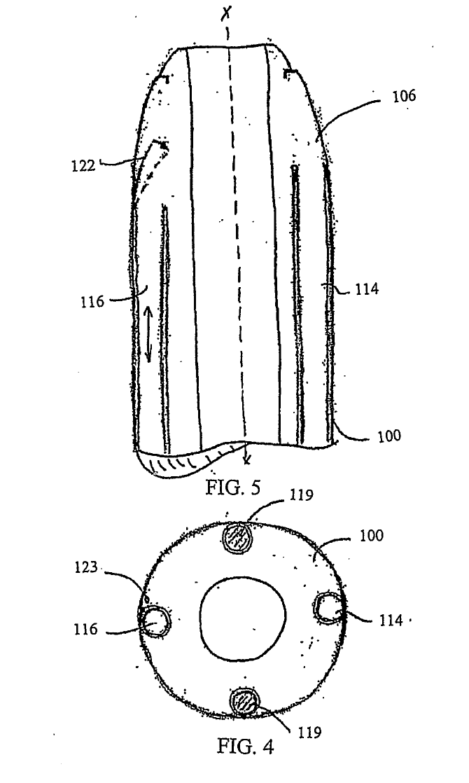

In one preferred embodiment, as shown in FIGS. 4 and 5, the overtube 100 includes two additional inner channels, namely a second inner channel 114 and a third inner channel 116, with proximal openings 115 and 117 covered by sealing caps 118 at the proximal end 104 and distal openings on the inner surface of the distal end 106 of the overtube 100. The overtube may define two additional stabilization channels 119 for stabilizing the overtube 100.

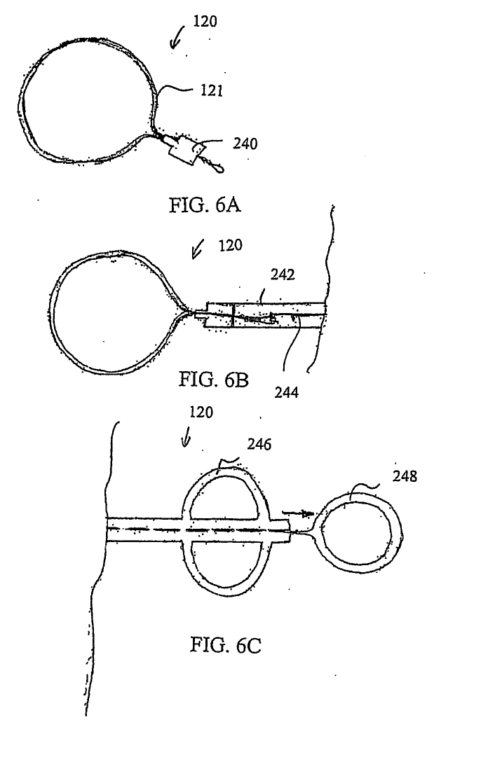

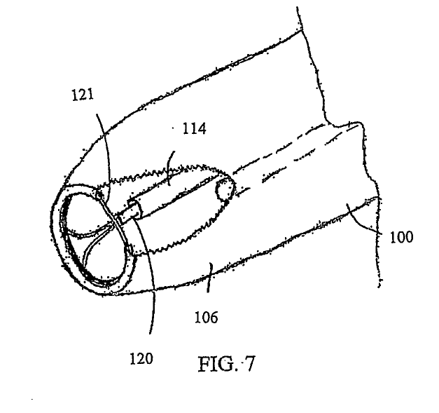

FIGS. 6A, 6B, and 6C illustrate a detachable ligating device 120 for ligating the appendix after the appendix has been inverted in accordance with one preferred embodiment of the present invention. The ligating device 120 includes a loop 121 at a distal end, a tight tube 240 received over an end portion of the loop 121, an elongated tube 242, and an elongated string or wire 244 passing through the elongated tube 242 and being attached to the loop 121. The loop 121 can be tightened by pushing the tight tube 240 over the loop 121 and pulling the elongated wire 244 through the handles 246 and 248. The elongated tube 242 maybe advanced into the second inner channel 114 of the overtube 100. The ligating device 120 is useful for ligating the vermiform appendix 20 when the appendix 20 is inverted into the cecum 12 and closing the cecal opening after the appendix 20 is cut, and preventing bleeding. The device 120 is preferably loaded into the second inner channel 114 before the procedure and the loop 121 is placed into an open-faced receiving channel at the distal end 106 of the overtube 100 before the procedure or after the overtube 100 is inserted into the colon, as shown in FIG. 7.

The third inner channel 116 is used for accommodating a cutting device, for example, a sphincterotome or a needle knife device, such as ZIMMON® sphincterotome and ZIMMON® needle knife. The sphincterotome or needle knife may be used to increase the diameter of the orifice 28 or a proximal portion of the central lumen 26 of the appendix 20 if it is necessary during the inversion procedure. As best shown in FIGS. 4 and 5, the third channel 116 is equipped with an angulation knob or a deflector 122 located at the distal end 106 of the overtube 100 and a wire 123 which extends through a channel between the proximal end and the distal end of the overtube 100. At the distal end 106, the wire 123 is connected to the knob 122 for adjusting the angle of the angulation knob 122 relative to the central axis of the channel 116. The angulation knob 122 may be used to change the angle of the cutting device at the distal end 106. The angulation knob 122 can be controlled by a surgeon from the proximal end of the overtube 100 through the wire 123. The channel 116 for receiving the cutting device and the angulation knob may be movable along the overtube 100 to enable cutting at several positions.

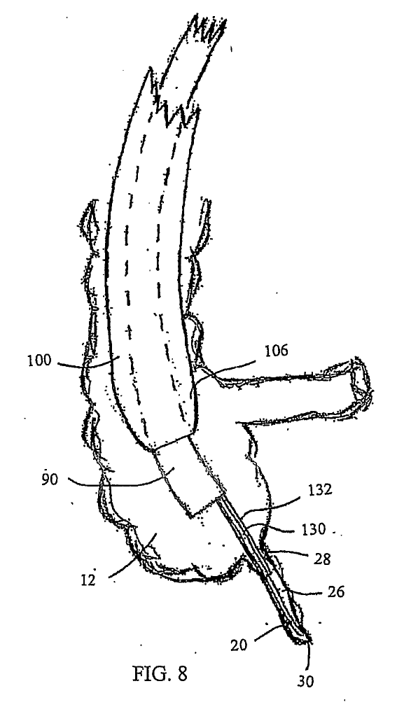

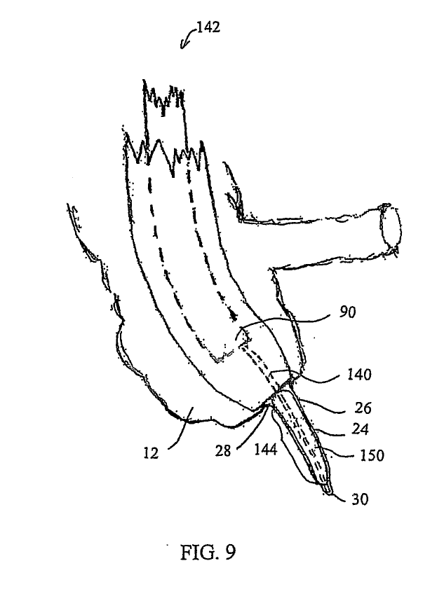

The colonoscope 90 may include at least one inner channel for passing surgical devices. In one preferred embodiment, the device for inversion and removal of a vermiform appendix further includes a guiding means, for example, a wire guide 130, which is adapted for passing through the inner channel of the colonoscope 90. In operation, as shown in FIG. 8, the wire guide 130 is advanced into the orifice 28 of the vermiform appendix 20 through the channel of the colonoscope 90. The position of the guiding wire 130 can be checked on a monitor using X-rays to determine the length and anatomic position of the appendix. A standard catheter 132 may be advanced over the guide wire 130 to irrigate the central lumen 26 of the vermiform appendix 20. The device further comprises an elongated flexible element 140, which extends between a proximal end 142 and a distal end 144, and is dimensioned to be received by the inner channel of the colonoscope 90 (FIG. 9). In one preferred embodiment, the elongated flexible element 140 maybe constructed as a catheter and the distal end 144 can be inserted into the central lumen 26 over the wire guide 130. An anchoring means 150 is attached to the distal end 144 of the elongated element 140 for anchoring the distal end 144 to the interior walls 24 of the central lumen 26 of the vermiform appendix 20. The anchoring means 150 include flaps, inflatable rubber tubes, grabbing means, vacuum means, or other suitable means which can increase the friction between the distal end 144 of the elongated element 140 and the interior walls 24 of the central lumen 26, or, which can grab or suck in the interior walls 24, which includes side walls of the central lumen 26 and the bottom wall at the distal tip 30 of the appendix 20. With that frictional engagement, when a surgeon pulls the elongated element 140 backward from the proximal end 142, the vermiform appendix 20 is pulled and inverted into the cecum 12 with the distal end 144 of the elongated element 140.

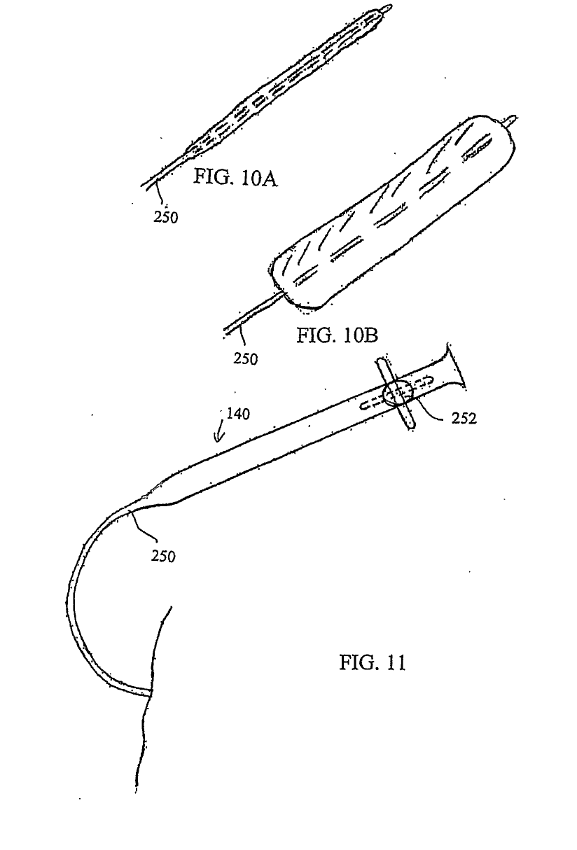

The elongated flexible element 140 and the anchoring means 150 may be customized for the length, diameter, location, form and flexibility of the vermiform appendix of a patient. Referring to FIGS. 10A, 10B, and 11, in one preferred embodiment, the elongated flexible element 140 and the anchoring means 150 are integrally constructed as a balloon catheter, which has an inflatable balloon 150 at the distal end 144 for anchoring the distal end 144 of the elongated element (catheter) 140 to the interior walls of the vermiform appendix. The balloon 150 is preferably inelastic so that, upon inflation, a specific contour is defined by the balloon 150. The balloon 150 is preferably cylindrical shaped when inflated (as shown in FIG. 10B), In a preferred form, the outer surfaces of the balloon 150 carry a skid resistant structure adapted to frictionally engage with the interior walls of the vermiform appendix in a highly effective manner. The balloon 150 can be inflated and deflated via a channel 250. The opening and closing of the channel can be controlled by a lock mechanism 252.

As shown in FIGS. 9-13, when the colonoscope 90 is placed in the cecum 12 and the distal end 144 of the catheter 140 is advanced into the central lumen 26 of the vermiform appendix 20, air is inflated into the balloon 150 through the channel 250 and the balloon 150 is inflated. The pressure inside the balloon 150 can be controlled with a manometer at the proximal end of the device. When the working pressure is reached, the channel 250 is locked by the lock 252.

Referring now to FIG. 8 to FIG. 13, the procedure starts with advancing an overtube 100 together with a colonoscope 90 into the cecum. The overtube 100 also can be advanced over the colonoscope 90 after the colonoscope 90 has been positioned within the colon. The distal end 106 of the overtube 100 is kept behind the tip of the colonoscope 90. Air or some other fluid medium is introduced into the colon through an instrument channel of the colonoscope 90 to insufflate the otherwise collapsed colon. A final position of the colonoscope 90 and the overtube 100 is reached when the distal end of the colonoscope 90 is placed in the cecum 12, and the sigmoid colon and the colonic flexures have been straightened. When the distal end of the colonoscope 90 is positioned within the cecum 12, as shown in FIG. 8, a wire guide 130 is advanced into the orifice 28 of the vermiform appendix 20 through an instrument channel of the colonoscope 90. The position of the wire guide 130 can be checked on a monitor using X-rays. A standard catheter 132 is advanced into the central lumen 26 of the appendix 20 to irrigate the central lumen 26. Feces remnants within the appendix 20 can be washed away by administering saline solution through a channel of the catheter 132. Feces which are washed out from the central lumen 26 may appear at the orifice 28 and can be later removed through the instrument channel of the colonoscope 90. Acute appendicitis can be diagnosed by the appearance of purulent material. When no more feces appears at the orifice 28 of the central lumen 26, a radiologically effective amount of contrast media can be placed over the catheter 132 and radiographs can be taken from the appendix 20. The diameter, length, and location of the vermiform appendix 20 can be seen during fluoroscope on an X-ray monitor. The vermiform appendix is inspected for signs of perforation. The mucous layer on the epithelial surface can also be washed away by using N-acetylcysteine.

Next, referring to FIG. 9, an elongated element 140, preferably a balloon catheter is advanced into the cecum 12. The distal end 144 of the catheter 140 is advanced into the central lumen 26 of the vermiform appendix 20. Preferably, the distal end 144 is advanced into the central lumen 26 over the guide wire 130.

If the orifice 28 or the proximal portion of the central lumen 26 of the vermiform appendix 20 shows a stricture or does not have an adequate diameter for inverting the appendix, they can be dilated using a balloon, for example, a QUANTUM TTC® balloon dilator. If using the balloon dilator does not produce a sufficient diameter, the mucosal, submucosal and muscular layer of the appendiceal orifice 28 and the proximal portion of the central lumen 26 can be cut by an appropriate cutting device. The cutting device may be similar to the type used for endoscopic sphincterotomy at endoscopic retrograde cholangio-pancreaticography (ERCP), such as a splincterotome or a needle knife. The cutting device is advanced into the cecum through the third inner channel 116 of the overtube 100. The angulation knob 122, which can be controlled by way of a wire 123 from the proximal end 104, is used to change the angle of the cutting device at the distal end 106. The channel for receiving the cutting device and the angulation knob may be moved along the overtube to enable cutting at several positions. To avoid opening of the peritoneal cavity, it is important to direct the cut into the mesoappendix. Although, the mesoappendix cannot be seen from the inside of the colon, its attachment to the appendix follows certain rules. First, the mesoappendix is a triangular fold of the peritoneum around the vermiform appendix, and is attached to the back of the lower end of the mesentery, close to the ileocecal junction. Second, from the lumen of the cecum it can be found when connecting the posterior lip of the ileocecal valve with the orifice of the appendix. Third, a proximal branch of the artery for the appendix can sometimes be seen piercing through the muscular layer at the side of the mesoappendix. Fourth, the posteromedial tenia coli can be used as a marker. It is not intended to cut the complete appendiceal wall, but only the mucosal, submucosal and parts of the muscular layer. The adequacy of the enlargement of the appendiceal orifice can be checked with a balloon dilator.

After the proximal portion of the appendix has been dilated, or the interior walls 24 have been partially cut, the distal end 144 of the elongated flexible element 140 is advanced into the appendiceal lumen 26 through the instrument channel of the colonoscope 90. The balloon 150 is inflated by air passing through the inner channel 250 of the catheter 140, and when the pressure inside the balloon 150 reaches a predetermined value, the channel 250 is locked by the lock 250.

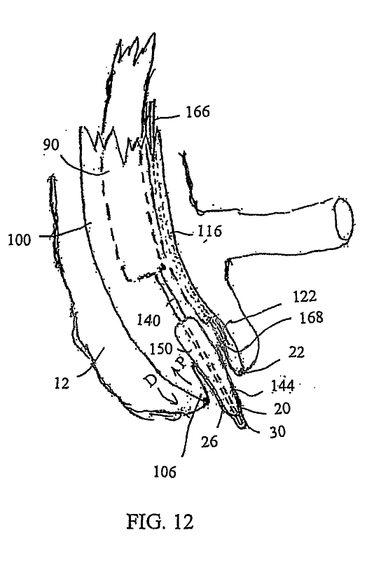

Referring to FIG. 12, then, the catheter 140 is pulled in a direction indicated by arrow P at the handle which locates at the proximal end of the colonoscope 90. The pulling force applied upon the interior walls 24 of the appendix by the anchoring means 150 at the distal end 144 of the catheter 140 draws the appendix into the cecum 12. In a preferred form, while the distal end 144 of the catheter 140 being pulled, the distal end 106 of the overtube 100 is advanced toward the appendix base 22 in a direction D opposite to the direction P, providing counterforce to the appendix base 22 around the orifice 28 of the appendix 20. The pulling force applied upon the interior walls 24 of the appendix and the counterforce applied upon the appendix base 22 cause the vermiform appendix 20 to be completely or partially inverted into the cecum 12, as illustrated in FIG. 13. An inversion that positions the distal tip 30 of the appendix 20 inside the orifice 28 of the appendix 20 is sufficient to advance a next step in the procedure.

During the inversion, the mesoappendix with the supplying artery is pulled into the space between the inverted and the non-inverted part of the appendix. This mesoappendix varies in diameter. A thick mesoappendix may cause the inversion to be incomplete. In such a case, the mucosal, submucosal and muscular layer at the inverted proximal part of the appendix can be cut by a tip 168 of a needle knife catheter 166 or a similar device that is placed in the second channel 116 of the overtube 100. The angulation knob 122 that is located at the outlet of the overtube channel 116 can be used to change the angle of the tip 168 at the distal end 106. The clockwise location of the needle knife can be adjusted by rotating the overtube 100. During the cutting, the pulling on the catheter 140 with the balloon 150 and pushing on the distal end 106 of the overtube 100 should be continued. As soon as the appendix can be further inverted, the cutting is finished. The incision is generally superficial and any opening of the peritoneal cavity should be avoided.

During the inversion of the vermiform appendix, a detachable ligating device 120 with a loop 121 shown in FIGS. 6A-6C, is placed at the orifice 28 of the vermiform appendix 20, which has the mesoappendix and the artery to the appendix at the outer surface of the inverted appendix. The ligating device 120, preferably a detachable ligating catheter, has been loaded in the first channel 114 of the overtube 100 before the procedure and held in a stable position at the distal end 106 of the overtube 100, as shown in FIG. 7.

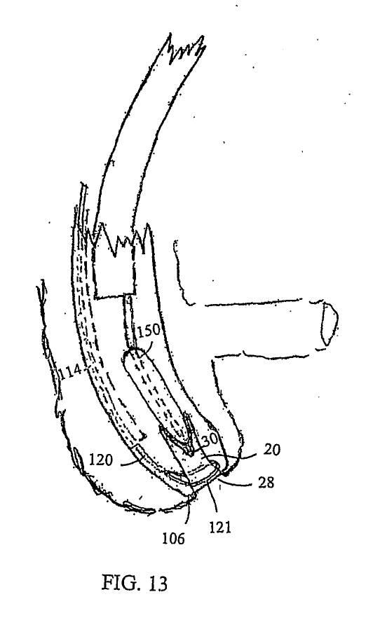

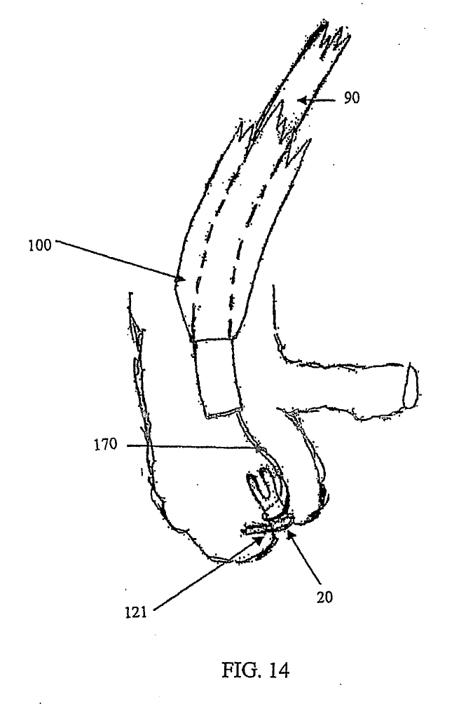

As shown in FIG. 13, the function of the ligating device 120 is to place a tight loop over the appendiceal orifice 28 for prevention of cecal leakage or bleeding from the artery to the appendix 20. After the detachable ligating device has been placed and secured, and the loop 121 has been detached from the catheter 120, the overtube can be partially retracted to allow a snare wire loop 170, like a polypectomy snare, which is illustrated in FIG. 14, to be placed over the inverted vermiform appendix 20 and to electrocut and electrocoagulate the appendix at a point close to the position where the loop 121 has been placed. Exemplary device of the snare wire loop 170 is snares or polyp retrievers, like ACUSNARE® snares and polyp retrievers. After the vermiform appendix is cut by the wire loop 170, the colonoscope 90 retrieves the removed vermiform appendix by pulling it through the overtube 100, which is kept in place and used for fast reinsertion of the colonoscope for the purpose of a second look at the site of colonoscopic appendectomy.

Many devices can be used to invert the appendix, like wire loops, tubes, stents, rubber bands, rubber balloons, clips and combinations thereof. These devices use mechanical pulling power or vacuum, or both to invert the appendix.

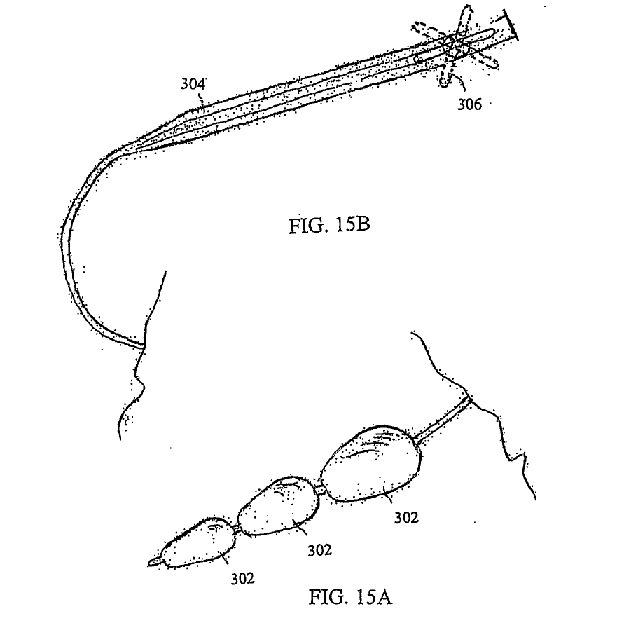

FIGS. 15A and 15B show an alternative form of the balloon catheter for inserting into the appendix and inverting the appendix. The device 300 includes a series of conical-shaped balloons 302, preferably three, each having the base of the cone at a proximal end of the balloon and the tip of the cone at a distal end of the balloon. The balloons 302 may be inflated and deflated simultaneously via a single channel, or as shown in FIG. 15B, are inflated and deflated separately and sequentially via three channels, which are defined in one elongated catheter 304. The inflating and deflating of the balloons 302 can be controlled by a lock mechanism 306, which can selectively control the three channels, at the proximal end of the elongated catheter 304.

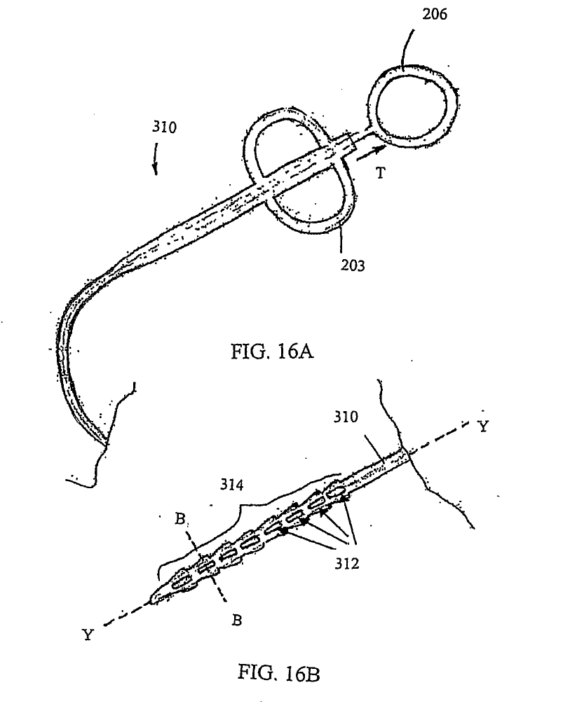

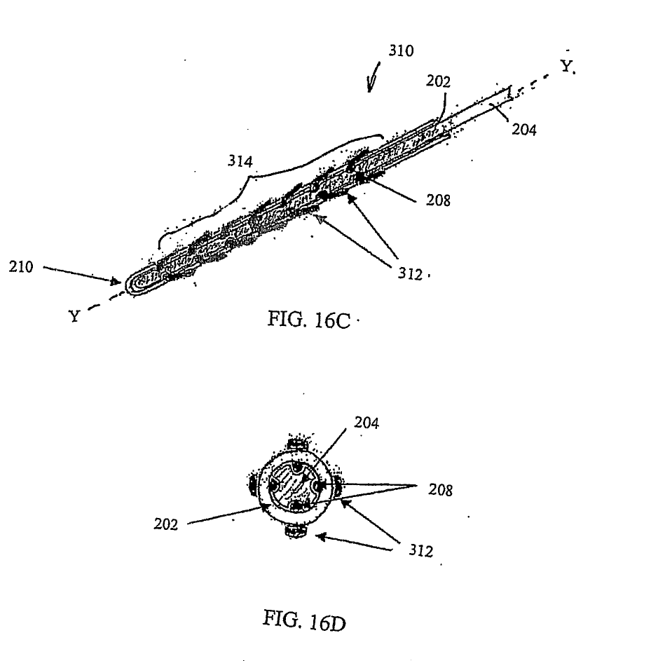

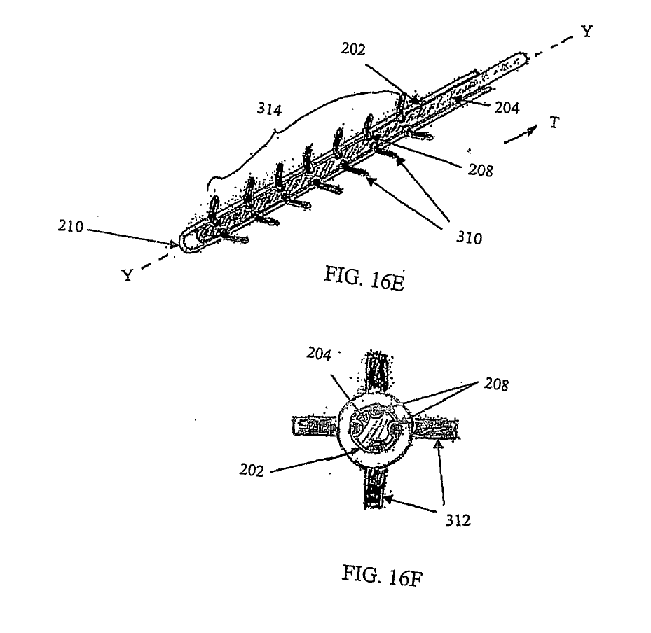

FIGS. 16A-16F illustrate another alternative embodiment of the elongated flexible element and anchoring means for inserting into the appendix and inverting the appendix. As shown in PIGS. 16A and 16B the elongated flexible element and anchoring means are constructed as a catheter 310 with adjustable flaps or spines 312 on the sides of the distal portion 314 of the catheter 310.

The catheter 310 includes a central lumen 202 and an inner operating element 204 which is received by the central lumen 202. The catheter 310 and the inner operating element 204 have handles 203 and 206 at the proximal end of the catheter 310. FIGS. 16C-16F are cross-sectional views of the distal end 314 of the catheter 310 and the adjustable flaps 312. The adjustable flaps 312 include knobs 208 extending into the central lumen 202, and the inner operating element 204 defines concave depressions for engaging with the knobs 208. As shown in FIGS. 16C and 16D, when the inner operating element 204 is advanced into the distal tip 210 of the distal end 314, the adjustable flaps 312 are positioned close to the outer surface of the distal end 314 of the catheter 310, and the distal end 314 can be inserted into the appendix. As shown in FIGS. 16E and 16F, after the distal end 314 is disposed within the appendix, the flaps are opened by pulling the inner operating element 204 backward, as shown by arrow T in FIG. 16E. The tips of the adjustable flaps 312 includes skid resistant structures that allow the flaps to grip the interior walls of the appendix. Then the catheter 310 with the inner operating catheter 204 is pulled into the cecum by pulling the handles 203 and 206. At the same time, the overtube is used to produce sufficient counterforce at the orifice of the appendix.

In alternative forms, the flaps may open in a sequential order, for example, the flaps at the base first and continuous opening up to the tip. The flaps may have different length, for example, longer at the base and smaller at the tip.

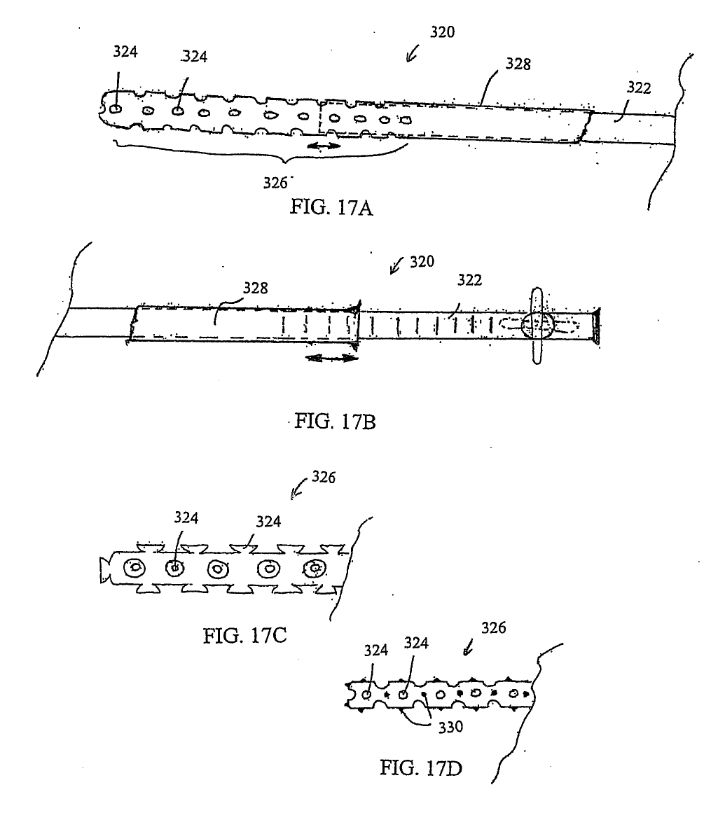

In another alternative embodiment, the device may use a vacuum for anchoring the distal end of the elongated flexible element to the interior walls of the appendix, and thereby inverting the appendix. As shown in FIGS. 17A through 17D, the vacuum device 320 comprises a catheter 322 with distal openings 324 at a distal portion 326 that will be introduced into the appendix. The material of the device has some flexibility but does not collapse when vacuum is produced. The vacuum may be produced at the handle outside of the patient with a syringe, at the proximal part of the device. Vacuum causes the wall of the appendix to attach to the catheter 322. When the device 320 is pulled for the purpose of inverting the appendix, the proximal openings would detach from the appendix wall. This would cause loosing the vacuum. An inner tube 328 is provided to move toward the tip of the device in order to close those openings 324, which are not anymore attached to the wall of the appendix during the inversion process. Thereby, the vacuum is kept within the appendix and the inversion can be completed.

In order to increase either the vacuum effect or the friction, the openings 324 of the vacuum device 320 may be shaped like octopus, or like suction cups (as shown in FIG. 17C), and the vacuum device may be provided with small rounded or pointy spines 330 on the outside of the distal part of the device 320 (as shown in FIG. 17D.), which anchor in the wall of the appendix.

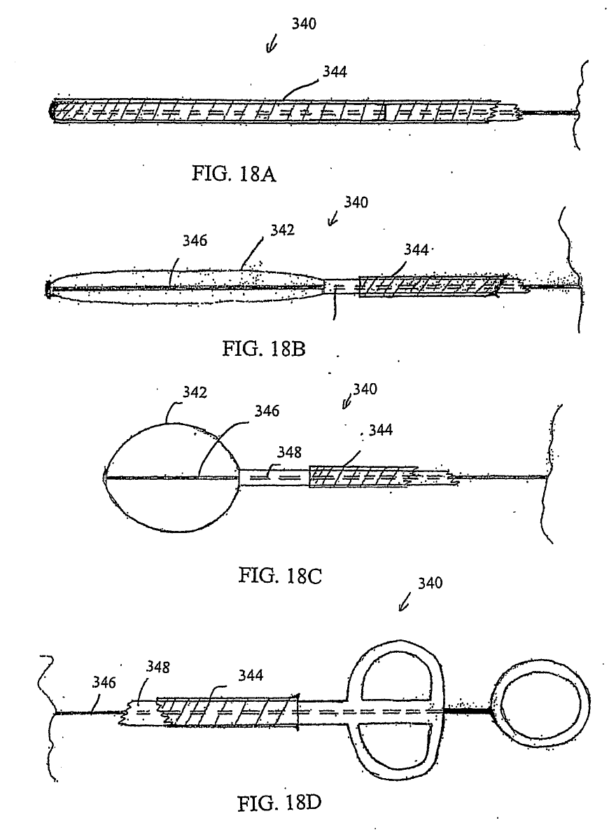

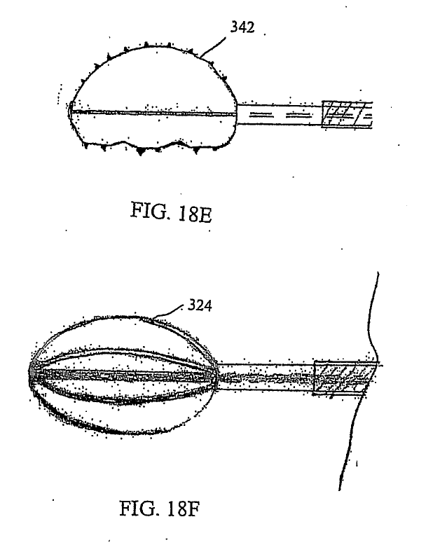

In another alternative form, as shown in FIGS. 18A through 18F, a set of wires is provided for anchoring the distal end of the elongated flexible element to the appendix walls and for inverting the appendix. The wire device 340 includes a set of wires 342 (at least one, but most likely four to eight), which are preferably covered by a cover sheet 344. The wires 342 are mounted on small inner catheter 346 and the distal ends of the wires 342 are fixed to the distal tip of the catheter 346. The proximal ends of the wires are fixed to a tube 348, which is positioned between the cover sheet 344 and the inner catheter 346. After the set of wires 342 is placed in the appendix, the cover sheet 344 is pulled off and the wires 342 are released. The cross section of the wires 342 is preferable flat, e.g. 1 mm ×0.3 mm, and the wires 342 can be preformed, thereby defining the final shape of the wire after released. When the inner catheter 346 is pulled against the middle tube 348, the wires 342 are pressed against the wall of the appendix. The appendix can be inverted by pulling the wires 342. As shown in FIG. 18E, the wires 342 may be provided with short rounded or pointy spines on the outer surface of the wires in order to increase friction. Also the shape and the number of the wires can be adapted to improve the friction/anchoring of wires in the wall of the appendix. FIG. 18F shows one preferred embodiment with four wires.

In another preferred embodiment, the wires 342 are self-expandable and are provided with specific waving pattern, e.g., a crisscross pattern to form a tubular mesh configuration, similar to an intravascular stent. The self-expandable force of such a device is variable. The tubular mesh (stent) is highly flexible in its longitudinal axis and is preferably covered with a lubricous sheet. After removing the cover sheet, it has a good expansible force in the radial direction, but there is a considerable (10 to 30%) shortening of the stent during release. For increasing friction, stents can also be combined with short rounded or pointy spines, which are mounted at the crossings of the wires.

The anchoring means may also employ combinations of the devices described above for anchoring the distal end of the elongated flexible element to the interior walls of the appendix, for example, combination of wire and balloon, combination of vacuum and balloon, combination of wire and vacuum, combination of wire, balloon, vacuum, and combination of flaps (or spines) and vacuum. The balloon can be one balloon or multi-balloon, and in the multi-balloon forms, the balloons can be inflated sequentially or simultaneously.

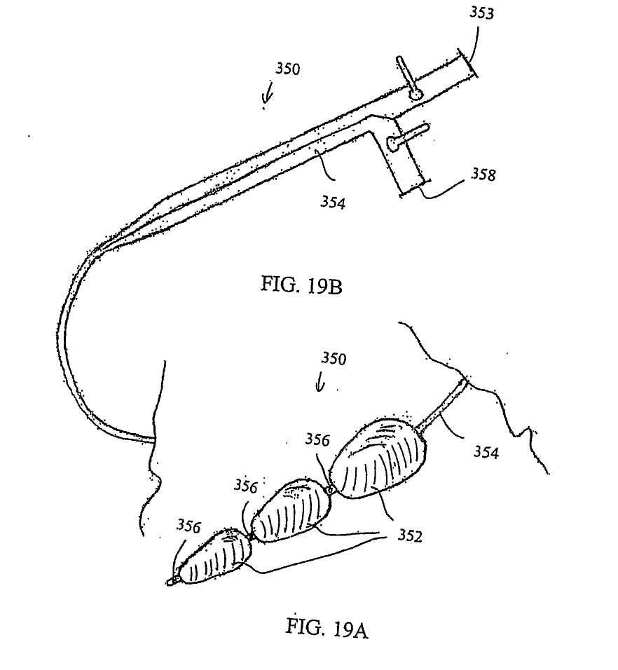

FIGS. 19A and 19B illustrate an embodiment indicated as 350 that employs a combination of balloon and vacuum. The balloons 352 may be connected over one inner channel 353 and be inflated or deflated simultaneously, or be connected over separated channels and be inflated or deflated separately and sequentially. Between the balloons 352 and at the distal tip of a catheter 354, are small holes 356 that are connected over a second inner channel 358 of the catheter 354. Channels 353 and 358 each have a lock disposed at the proximal end of the catheter 354 for locking the channels. The small openings 356 and the second channel 358 are used to produce vacuum inside the appendix. In the procedure, when the pressure inside the balloons 352 reaches a predetermined value, a vacuum can be produced through the openings 356 and second channel 358 by a syringe disposed at the proximal opening of the second channel 358.

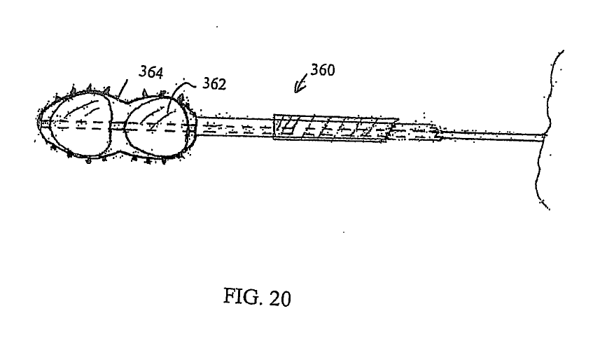

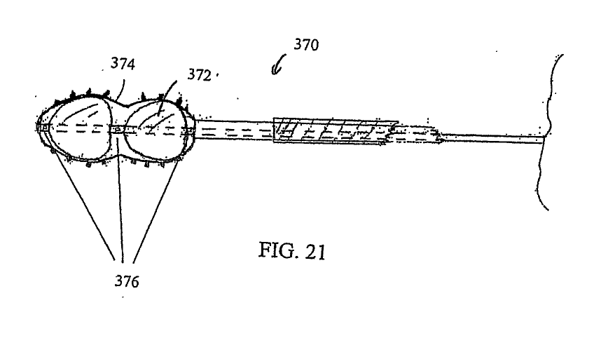

FIG. 20 shows a combination of wires and balloons for anchoring to the interior walls of the appendix and inverting the appendix. The device 360 includes balloons 362 and wires 364 having spines on the outer surfaces of the wires. FIG. 21 shows a combination of wires, balloons, and vacuum. The devices 370 includes balloons 372, wires 374, and vacuums holes 376.

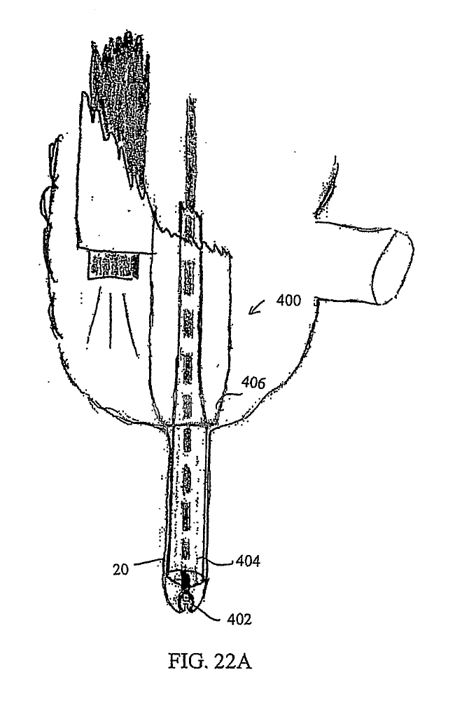

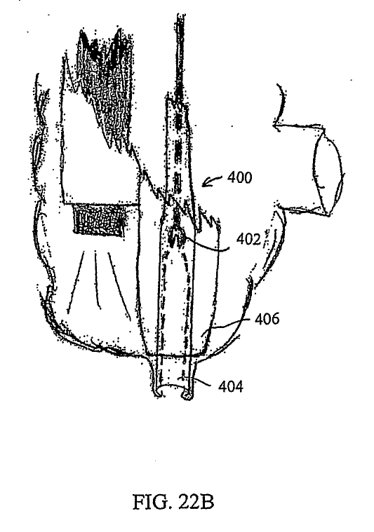

The above described inversion devices are preferably used in procedures performing inversion from the base of the appendix to the tip of appendix. The present invention also provides a tip-to-base inversion device 400, which, as shown in FIGS. 22A and 22B, may include an inner grabbing device 402, such as a forceps or the like, a sliding tube 404 received over the grabbing device 402, and an overtube 406 received over the sliding tube 404. In the procedure, the grabbing device 402 and the sliding tube 404 are advanced into the appendix 20. The grabbing device 402 grabs the tip of the appendix 20 and withdraw the tip into the sliding tube 404. The sliding tube 404 ensures that the wall of the appendix does not wrinkle up when the tip of the appendix is pulled during the inversion process.

The overtube 406 is placed at the orifice of the appendix for applying couterforce to the base of the appendix. As shown in FIG. 22B, the sliding tube 404 would slide into the overtube 406 as a consequence of pulling the inner grabbing device 402 and holding the overtube 406 at the base of the appendix during the inversion. The diameter and length of the sliding tube can be customized.

In an alternative form, the sliding tube 404 may be constructed as a coated, self-expandable mesh wire stent, for example, similar to an intravascular stent. The coat is of a flexible, lubricous material in order to slide within the appendix. The coated self-expandable mesh wire stent has at least the length of the appendix. It is released from an introducing tube by withdrawing a cover sheet. The advantage of such a coated, self-expandable mesh wire is that the expansion increases the space, into which the tip of the appendix is pulled.

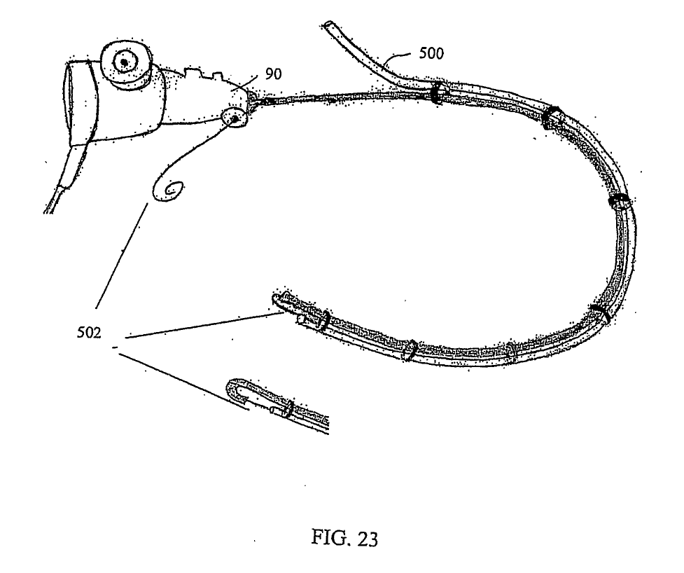

As shown in FIG. 23, in another preferred embodiment, the devices for performing the appendectomy, for example, balloons, wires, and etc., may be introduced into the cecum not over the instrument channel of a colonoscope but independent of it. The colonoscope 90 is only used for maneuvering the independent devices (indicated as 500) into the final position and for visual control during the procedure. The independent device 500 may be attached to the colonoscope 90 by string, bands, clips or by using a double overtube.

A string 502 connects the tip of the independent device 500 (which may includes an overtube that will be used for passing through and holding devices for appendectomy) with an instrument channel of the colonoscope 90. By using the string 502, the independent device 500 can be dragged behind the colonoscope and safely moved to the caecum. If needed, clips, bands or similar can attach the independent device 500 to the colonoscope 90.

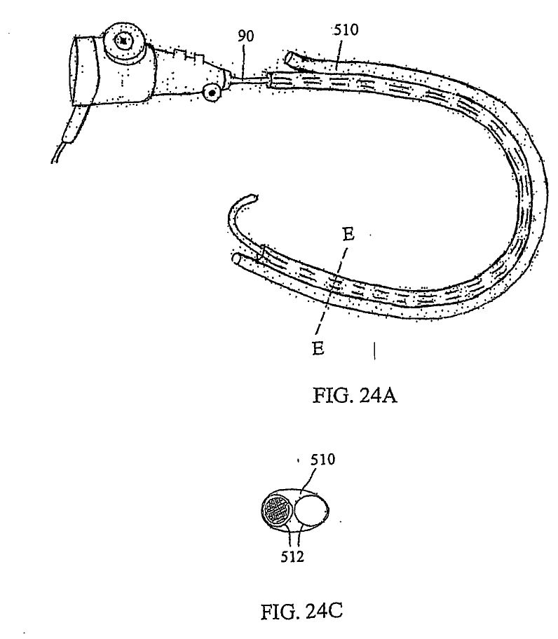

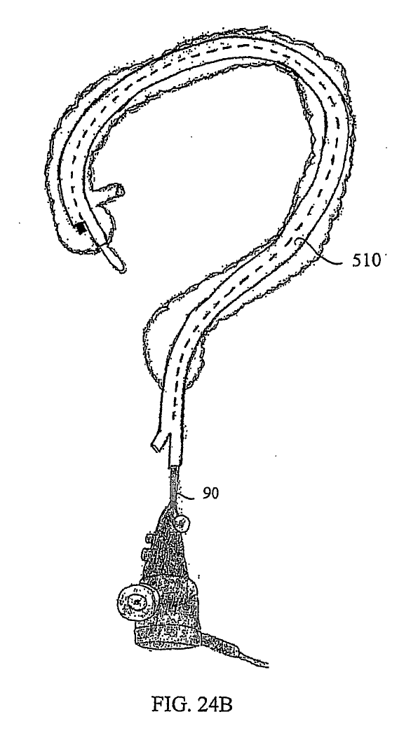

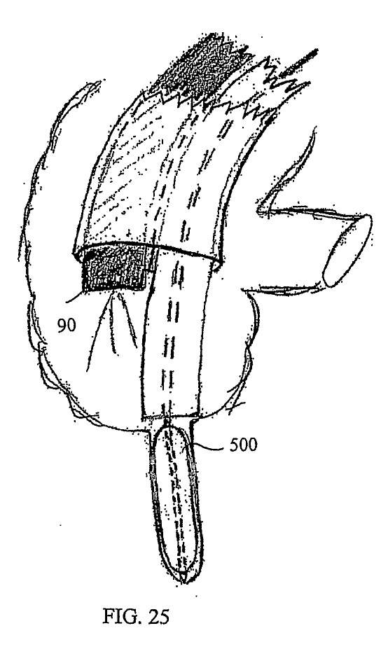

Similar functionality is achieved in embodiments having a double overtube 510, as shown in FIGS. 24A to 24C, that allows the advancement of either two colonoscopes 90 or one colonoscope (for maneuver control and visualization) and one independent device (for actually performing the endoscopic appendectomy or for other interventions in the colon that need larger instrument channels). The length of two overtubes is variable and may be different. The two inner tubes 512 can be moved independently back and forward within the outer tube 510. The independent devices, which can be introduced into the cecum by binding with the colonoscope, are not limited by the diameter of the instrument channel of the colonoscope (in general 3.2 mm). All devices as the ones described above, for example, balloons, catheter with spines or flaps, wires, vacuums, and the combinations thereof, and the grabbing device 400 shown in FIGS. 22A and 22B, can be adapted for use as a colonoscope independent device (as shown in FIG. 25).

Another advantage is that the device inverts the appendix is in the center of the tube through which the independent device passes, and the tube provides the counterforce.

This improves the pulling/pushing efficiency. If the devices are advanced through the instrument channel of the colonoscope, since the instrument channel of the colonoscope is typically not in the center of the colonoscope, the pulling during the inversion is outside of the center of the tube, which provides the counterforce. The features of the double overtube can be similar as described for the single overtube, holding a detachable ligating device (similar to Endloop®), and cutting devices such as a needle knife, sphincterotome or polypectomy snare. As with the single overtube, the number of instrument channels within the overtube can be higher than two. The distal end of the overtube can be constructed of a transparent material in order to better observe the inversion process with the colonoscope.

In the same regard, triple overtube or quadruple overtube, or more can be used. The channels inside the tubes may have different diameters and be used for different purposes.

In another preferred embodiment, the device for performing colonoscopic appendectomy includes two colonoscope, a first one used for operating the elongated flexible element, a second one for providing counterforce at the base of the appendix, instead of the overtube. The second colonoscope can be constructed like a overtube having a central lumen to accommodate the first colonoscope. In another preferred embodiment, the device may include only one colonoscope that has at least two instrument channels, a first one for receiving the elongated element and a second one for receiving means for providing counterforce to the base of the appendix. The means for providing counterforce can be a ring or tube, or other suitable devices, which can be operated through a wire passing through the second instrument channel. The ring or tube can be disposed at the distal end of the colonoscope before the procedure starts and can be. advanced toward the appendix base by way of the wire. In these two embodiments, the colonoscopes may have more additional inner channels for passing through ligating means, needle knife, snare wire, and other instrument that is useful during the-operation.

The device for performing the inversion appendectomy may also employ an overtube with a steering device only, but not a colonoscope. The surgeon can steer the distal end of the overtube to the cecum by using the steering device. The inversion devices including balloons, wires, or vacuum, and ligating and cutting devices then can pass through the overtube to the appendix.

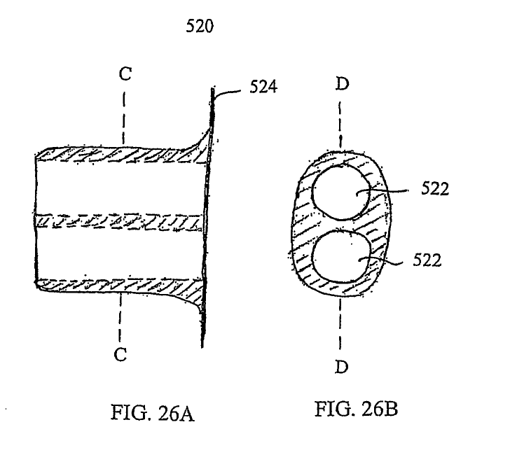

As shown in FIGS. 26A and 26B, the device may further comprise an anal adapter 520 for passage of the anal canal with two colonoscopes or one colonoscope and one independent device. The anal adapter 520 is preferably covered with soft sponge like material with two channels 522 for introduction of the colonoscopes or one colonoscope and one independent device. The adapter 520 may include a plate 524 to prevent that the adapter 520 is lost in the anus. The shape and the number of channels can be adapted to specific needs. Colonoscopic appendectomy is a safe procedure and can be done on ambulatory basis. It does not require anesthesia, does not leave skin scars, and the trauma and the wound healing are less.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description. All changes that come within the meaning and range of the equivalency of the claims are therefore intended to be embraced therein.

1. A kit for performing an inversion of a vermiform appendix of a patient, said appendix including interior walls defining a central lumen extending from a base which is connected to a cecum of the patient toward a distal tip of said appendix, said kit comprising:
a tube extending between a proximal end and a distal end, said tube defining at least one inner channel, said distal end being adapted to be inserted into a colon of a patient whereby said distal end is proximal to said appendix;
an elongated flexible element extending between a proximal end and a distal end, said elongated flexible element being adapted for passing through an said inner channel;
means disposed at or near said distal end of said elongated flexible element for anchoring said distal end of said elongated flexible element to at least one of said interior walls when said distal end is disposed within said central lumen, such that pulling said elongated flexible element backward provides force for inverting said vermiform appendix into said cecum; and
means for providing counterforce against said appendix base, wherein said means for providing counterforce against said appendix base is adapted for being advanced over said elongated flexible element toward said appendix base and engaging with said appendix base.

2. A kit for performing an inversion of a vermiform appendix of a patient according to claim 1, wherein said elongated flexible element is an elongated flexible catheter.

3. A kit for performing an inversion of a vermiform appendix of a patient according to claim 1 further comprising a guiding means for guiding said distal end of said elongated flexible element into said central lumen of said appendix.

4. A kit for performing an inversion of a vermiform appendix of a patient according to claim 3, wherein said guiding means comprises a wire guide, and wherein said elongated flexible element is a catheter disposed over said wire guide.

5. A kit for performing an inversion of a vermiform appendix of a patient according to claim 1, wherein said means disposed at said distal end of said elongated flexible element for anchoring said distal end of said elongated flexible element to at least one of said interior walls when said distal end is disposed within said central lumen comprises at least one balloon attached to said distal end of said elongated flexible element, and wherein said elongated flexible element defines a first inner channel in connection with an interior area of said at least one balloon for inflating and deflating said at least one balloon.

6. A kit for performing an inversion of a vermiform appendix of a patient according to claim 5, wherein said at least one balloon has an outer surface carrying a skid resistant structure.

7. A kit for performing an inversion of a vermiform appendix of a patient according to claim 5, wherein said elongated flexible element defines a second inner channel, said second inner channel having at least one distal opening at the distal end of the elongated element.

8. A kit for performing an inversion of a vermiform appendix of a patient according to claim 5, wherein said at least one balloon comprises a plurality of balloons connected by said first inner channel, and wherein said elongated flexible element defines a second inner channel which includes at least one opening located between any two adjacent balloons.

9. A kit for performing an inversion of a vermiform appendix of a patient according to claim 1 further comprising a colonoscope defining an inner channel for receiving said elongated flexible element.

10. A kit for performing an inversion of a vermiform appendix of a patient according to claim 1 further comprising a catheter being positionable within an inner channel of a colonoscope for irrigating said central lumen of said appendix.

11. A kit for performing an inversion of a vermiform appendix of a patient according to claim 1 wherein said means for providing counterforce against said appendix base comprises an elongated flexible tubular element extending between a proximal end and a distal end, said tubular element defining at least one inner channel, said at least one inner channel comprising a first inner channel which is adapted for receiving a colonoscope.

12. A kit for performing an inversion of a vermiform appendix of a patient according to claim 11, wherein said tubular element further comprises a circular seal disposed within said first inner channel at or near said proximal end of said tubular element, and wherein when a colonoscope is disposed within said first inner channel, said circular seal engages with said colonoscope and seals a region between said colonoscope and said tubular element.

13. A kit for performing an inversion of a vermiform appendix of a patient according to claim 11, wherein said first inner channel is coated with a lubricious material.

14. A kit for performing an inversion of a vermiform appendix of a patient according to claim 1 further comprising a ligating means being positionable near said appendix base for ligating said appendix when said appendix is disposed within said cecum.

15. A kit for performing an inversion of a vermiform appendix of a patient according to claim 14, wherein said ligating means comprises a loop at a distal end.

16. A kit for performing an inversion of a vermiform appendix of a patient according to claim 15, wherein said loop is detachable.

17. A kit for performing an inversion of a vermiform appendix of a patient according to claim 1 further comprising a cutting device for enlarging an orifice of said central lumen of said appendix.

18. A kit for performing an inversion of a vermiform appendix of a patient according to claim 17, wherein said cutting device comprises a sphincterotome.

19. A device for performing an inversion of a vermiform appendix of a patient according to claim 17, wherein said cutting device comprises a needle knife.

20. A kit for performing an inversion of a vermiform appendix of a patient according to claim 1 further comprising means for cutting said appendix when said appendix is disposed within said cecum.

21. A kit for performing an inversion of a vermiform appendix of a patient according to claim 20, wherein said cutting means comprises an elongated wire forming a wire loop at a distal end, said wire being electrically conductive.

22. A kit for performing an inversion of a vermiform appendix of a patient according to claim 1, wherein said means disposed at said distal end of said elongated flexible element for anchoring said distal end of said elongated flexible element to at least one of said interior walls when said distal end is disposed within said central lumen comprises at least one flap disposed at said distal end, said at least one flap being adjustable between a closed position and an opened position.

23. A kit for performing an inversion of a vermiform appendix of a patient according to claim 22, wherein said elongated flexible element comprises a catheter defining a central lumen and an inner elongated element slidingly received within said central lumen, said inner elongated element defining at least one depression, and wherein said at least one flap comprises a knob extending into said central lumen, said depression engaging with said knob, wherein said at least one flap are adjusted between said closed position and said opened position by operating said inner elongated element.

24. A kit for performing an inversion of a vermiform appendix of a patient according to claim 1, wherein said means for providing counterforce against said appendix base comprises a colonoscope, said colonoscope having a distal end being adapted for providing counterforce against said appendix.

25. A kit for performing an inversion of a vermiform appendix of a patient according to claim 1, wherein said means for providing counterforce against said appendix base comprises a ring, wherein said ring is adapted for being advanced over said elongated flexible element toward said appendix base and engaging with said appendix base.

26. A kit for performing removal of a vermiform appendix from a patient, said appendix including interior walls defining a central lumen extending from a base which is connected to a cecum of the patient toward a distal tip of said appendix, said kit comprising:
A. a tubular element extending between a proximal end and a distal end thereof, said tubular element defining at least one inner channel, wherein said at least one inner channel comprises a first inner channel being adapted for accommodating said flexible element;
B. an elongated flexible element extending between a proximal end and a distal end, said elongated flexible element being adapted for passing through said inner channel; and
C. means disposed at or near said distal end of said elongated flexible element for anchoring said distal end to at least one of said interior walls when said distal end is disposed within said central lumen.

27. A kit for performing removal of a vermiform appendix from a patient according to claim 26 further comprising means being positionable at the distal end of said tubular element for ligating said appendix when said appendix is disposed within said cecum of the patient.

28. A kit for performing removal of a vermiform appendix from a patient according to claim 27, wherein said means for ligating said appendix comprises a loop at a distal end.

29. A kit for performing removal of a vermiform appendix from a patient according to claim 28, wherein said tubular element further defines a second inner channel for receiving said means for ligating said appendix.

30. A kit for performing removal of a vermiform appendix from a patient according to claim 26, wherein said means disposed at said distal end of said elongated flexible element for anchoring said distal end to said interior walls when said distal end is disposed within said central lumen comprises at least one adjustable flap mounted on said distal end of said elongated flexible element.

31. A kit for performing removal of a vermiform appendix from a patient according to claim 26, wherein said means disposed at said distal end of said elongated flexible element for anchoring said distal end of said elongated flexible element to at least one of said interior walls when said distal end is disposed within said central lumen comprises at least one balloon attached to said distal end of said elongated flexible element, and wherein said elongated flexible element defines a first inner channel in connection with an interior area of said at least one balloon for inflating and deflating said at least one balloon.

32. A kit for performing removal of a vermiform appendix from a patient according to claim 31, wherein said at least one balloon comprises an outer surface carrying a skid resistant structure.

33. A kit for performing removal of a vermiform appendix from a patient according to claim 26 further comprising means being positionable at said appendix base for cutting said appendix.

34. A kit for performing removal of a vermiform appendix from a patient according to claim 33, wherein said means for cutting said appendix comprises an elongated wire forming a wire loop at a distal end, and wherein said elongated wire is electrically conductive.

35. A kit for performing removal of a vermiform appendix from a patient according to claim 26, wherein said at least one inner channel comprises a second inner channel having a distal opening at or near the distal end of the tubular element for receiving a cutting device for enlarging an orifice of the central lumen of the appendix, and wherein said tubular element comprises a knob disposed at or near the distal opening of the second inner channel for adjusting the cutting device.

36. A kit for performing removal of a vermiform appendix from a patient, said appendix including interior walls defining a central lumen extending from a base which is connected to a cecum of the patient toward a distal tip, said kit comprising:
A. a tube extending between a proximal end and a distal end, said tube defining at least one inner channel, said distal end being adapted to be inserted into a colon of a patient whereby said distal end is proximal to said appendix;
B. an elongated flexible element extending between a proximal end and a distal end, said elongated flexible element being adapted to be received within said inner channel, said distal end being dimensioned to fit within said central lumen;
C. means attached at or near said distal end of said elongated flexible element for anchoring said distal end to said interior walls when said distal end is disposed within said central lumen;
D. a tubular element extending between a proximal end and a distal end thereof, said tubular element defining an inner channel, wherein said distal end of said tubular element is dimensioned to interferingly engage with said appendix base to provide counterforce against said appendix base during an inversion of said appendix; and
E. means being positionable at said distal end of said tubular element for ligating said appendix when said appendix is disposed within said cecum of the patient.

37. A kit for performing removal of a vermiform appendix from a patient according to claim 36, wherein said means being positionable at said distal end of said tubular element for ligating said appendix when said appendix is disposed within said cecum of the patient comprises a loop at a distal end of said means for ligating said appendix.

38. A kit for performing removal of a vermiform appendix from a patient according to claim 36 further comprising cutting means being positionable at said appendix base for cutting said appendix.

39. A kit for performing removal of a vermiform appendix from a patient according to claim 38, wherein said cutting means comprises an elongated wire forming a wire loop at a distal end, and wherein said wire is electrically conductive.

40. A kit for performing removal of a vermiform appendix from a patient according to claim 36 further comprising a colonoscope, wherein said colonoscope defines an inner channel for receiving said elongated flexible element, and wherein said colonoscope is adapted for passing through the inner channel of the tubular element.

41. A method for performing an inversion of a vermiform appendix of a patient, said appendix including interior walls defining a central lumen extending from a base which is connected to a cecum of the patient toward a distal tip, said method comprising:
inserting a distal end of an elongated flexible element into said central lumen of said appendix;
anchoring the distal end of the elongated flexible element to at least one of said interior walls of said central lumen of said appendix; and
pulling said distal end of said elongated flexible element toward said cecum, wherein said distal end of said elongated flexible element draws said appendix into said cecum.

42. A method for performing an inversion of a vermiform appendix of a patient according to claim 41 further comprising providing a counterforce to said appendix base.

43. A method for performing an inversion of a vermiform appendix of a patient according to claim 41 further comprising irrigating said central lumen of said appendix before said distal end of said elongated flexible element is inserted into the central lumen of the appendix.

44. A method for performing an inversion of a vermiform appendix of a patient according to claim 41 wherein the step of inserting a distal end of an elongated flexible element into said central lumen of said appendix comprises:
inserting an introducing means into said central lumen of said appendix; and
guiding a distal end of an elongated flexible element into said central lumen of said appendix over said introducing means.

45. A method for performing an inversion of a vermiform appendix of a patient according to claim 41, wherein said elongated flexible element comprises:
means disposed at said distal end of said elongated flexible element for anchoring said distal end of said elongated flexible element to at least one of said interior walls when said distal end is disposed within said central lumen.

46. A method for performing an inversion of a vermiform appendix of a patient according to claim 41 further comprising ligating said appendix after said appendix is drawn into said cecum.

47. A method for performing an inversion of a vermiform appendix of a patient according to claim 46 further comprising cutting said appendix after said appendix is ligated.

48. A method for performing an inversion of a vermiform appendix of a patient according to claim 41 further comprising cutting said appendix after said appendix is drawn into said cecum.

49. A method for performing an inversion of a vermiform appendix of a patient according to claim 41 further comprising placing a colonoscope into said cecum before said distal end of said elongated flexible element is inserted into said central lumen of said appendix.

50. A method for performing removal of a vermiform appendix from a patient, said appendix including interior walls defining a central lumen extending from a base which is connected to a cecum of the patient toward a distal tip, said method comprising:
guiding a distal end of an elongated flexible element into the central lumen of the appendix, wherein said elongated flexible element comprises means attached to said distal end of said elongated flexible element for anchoring said distal end of said elongated flexible element to at least one of said interior walls of said central lumen when said distal end of said elongated flexible element is disposed within said central lumen;
anchoring said distal end of said elongated flexible element to at least one of said interior walls of said central lumen by said means for anchoring said distal end of said elongated flexible element to at least one of said interior walls of said central lumen;
advancing a distal end of a tubular element toward said appendix base to provide counterforce against said appendix base; and
pulling said distal end of said elongated flexible element toward said cecum, wherein said elongated flexible element draws said appendix into said cecum.

51. A method for performing removal of a vermiform appendix from a patient according to claim 50 further comprising ligating said appendix after said appendix is drawn into said cecum.

52. A method for performing removal of a vermiform appendix from a patient according to claim 51 further comprising cutting said appendix after said appendix is ligated.

53. A method for performing removal of a vermiform appendix from a patient according to claim 50 further comprising cutting said appendix after said appendix is drawn into said cecum.

54. A method for performing an inversion of a vermiform appendix of a patient according to claim 50 further comprising irrigating said central lumen of said vermiform appendix before said distal end of said elongated flexible element is guided into said central lumen.

55. A method for performing removal of a vermiform appendix from a patient according 50, wherein said means for anchoring said distal end of said elongated flexible element to at least one of said interior walls of said central lumen comprises at least one balloon attached to said distal end of said elongated flexible element.

56. A method for performing removal of a vermiform appendix from a patient according 50, wherein said means for anchoring said distal end of said elongated flexible element to at least one of said interior walls of said central lumen comprises at least one adjustable flap attached to said distal end of said elongated flexible element.

57. A method for performing removal of a vermiform appendix from a patient according 50 further comprising placing a colonoscope into the cecum before said distal end of said elongated flexible element is guided into said central lumen.

58. A method for performing removal of a vermiform appendix from a patient, said appendix including interior walls defining a central lumen extending from a base which is connected to a cecum of the patient toward a distal tip, said method comprising:
placing a colonoscope into said cecum of the patient;
guiding a distal end of an elongated flexible element into the central lumen of the appendix;
anchoring said distal end of said elongated flexible element to at least one of said interior walls of said central lumen;
providing counterforce against said appendix base;
pulling said elongated flexible element backward into said cecum, wherein said elongated element draws said appendix into said cecum; and
ligating said appendix.

59. A method for performing removal of a vermiform appendix from a patient according to claim 58 further comprising cutting said appendix after ligating said appendix.

60. A method for performing an inversion of a vermiform appendix of a patient according to claim 58 further comprising irrigating said central lumen of said vermiform appendix before said distal end of said elongated flexible element is guided into said central lumen.

61. A method for performing imaging of a vermiform appendix of a patient, said method comprising:
placing a radiologically effective amount of contrast media into the vermiform appendix; and
inspecting the vermiform appendix by radiography.

62. A method for performing imaging of a vermiform appendix of a patient according to claim 61 further comprising irrigating said vermiform appendix before said radiologically effective amount of contrast media is placed into the vermiform appendix.

63. A method for performing imaging of a vermiform appendix of a patient according to claim 61 further comprising placing a colonoscope into a cecum of the patient before said radiologically effective amount of contrast media is placed into the vermiform appendix.
